(19) United States
(12) Patent Application Publication
(10) Pub. No.: US 2023/0175048 A1
(43) Pub. Date: Jun. 8, 2023

(54) METHODS FOR SIMULTANEOUSLY DETECTING TARGET NUCLEIC ACIDS AND PROTEINS AND A KIT THEREOF

(71) Applicant: ADVANCED CELL DIAGNOSTICS, INC., Newark, CA (US)

(72) Inventors: Xiao-Jun Ma, Newark, CA (US); Bingqing Zhang, Newark, CA (US); Courtney Todorov, Newark, CA (US); Lydia Hernandez, Newark, CA (US)

(21) Appl. No.: 17/998,113

(22) PCT Filed: May 6, 2021
(86) PCT No.: PCT/US2021/031034

§ 371 Date: Nov. 7, 2022

Related U.S. Application Data
(60) Provisional application No. 63/021,632, filed on May 7, 2020.

(51) Int. Cl. C12Q 1/6841; C12Q 1/6806; G01N 33/58

(52) U.S. Cl. CPC C12Q 1/6841; C12Q 1/6806; G01N 33/582; G01N 2474/20

(57) ABSTRACT

A method of simultaneously detecting target nucleic acids and target proteins in a biological sample, comprising treating the biological sample with a crosslinking agent, that is after incubating it with a primary antibody that detects the target proteins and prior to detecting the target nucleic acids by in situ hybridization.

### CROSS REFERENCE TO RELATED APPLICATIONS

This application claims priority to and the benefit of U.S. Provisional Pat. Application No. 63/021,632 filed May 07, 2020, which is incorporated herein by reference in its entirety and for all purposes.

### FIELD

Embodiments of the present disclosure include methods for preparing a biological sample for simultaneously detecting a target nucleic acid and a target protein. Methods for simultaneously detecting a target nucleic acid and a target protein, as well as kits for carrying out the methods are also provided.

### BACKGROUND

In situ hybridization (ISH) is a molecular biology technique widely used to detect and localize specific sequences within cells or tissue sections while preserving the cellular and tissue context. ISH, therefore, enables spatial-temporal visualization as well as quantification of gene expression within cells and tissues, which has useful applications in research and in diagnostics. See Hu et al., Biomarker Research 2(1): 1-13 (2014); Ratan et al., Cureus 9(6): e1325 (2017); Weier et al., Expert Review of Molecular Diagnostics 2(2): 109-119 (2002).

Immunohistochemistry (IHC) and immunocytochemistry (ICC) are also powerful techniques that are used to detect and localize specified proteins within tissue sections and cells, while similarly maintaining spatial resolution and cytological context. Similar to ISH, IHC and ICC have broad and complementary applications in research and diagnostics. See Shi et al., Journal of Histochemistry & Cytochemistry 59(11): 13-32 (2011). For example, both approaches provide researchers insights regarding cell identities and states.

Complete characterization of complex cellular interactions within a tissue or cell requires a multi-omic strategy. For example, detecting and analyzing transcriptomic and proteomic information is informative in interrogating complex tissues and in revealing cell-type specific gene expression (see Vanlandewijck et al., Nature 554(7693): 475-482 (2018); Stempl et al., the Journal of Molecular Diagnostics 14(1): 22-29 (2014)), in identifying the cellular sources of secreted proteins (see Liou et al., Cell Reports 19(7): 1322-1333 (2017)), and in visualizing the spatial organization of the various cell types and their interactions. To fully and accurately characterize cells and tissues, both nucleic acids and proteins need to be detected simultaneously in the same tissue sample in a spatially resolved manner.

Despite the critical roles of dual detection, it has been problematic to obtain high-quality signals for both nucleic acids and proteins in the same sample. A significant demand therefore exists for new methods that can detect both signals, for example, performing ISH and IHC/ICC simultaneously in one sample without interference from each other. The present disclosure addresses these and other needs.

### 4. SUMMARY

In one aspect, provided herein is a method for preparing a biological sample for simultaneously detecting a target nucleic acid and a target protein, comprising (i) incubating the biological sample with a primary antibody; (ii) treating the biological sample with a crosslinking agent after (i); and (iii) detecting the target nucleic acid by in situ hybridization after (ii).

In some embodiments, the method further comprises treating the biological sample with a protease after treating the biological sample with the crosslinking agent and before detecting the target nucleic acid by in situ hybridization. In other embodiments, the method further comprises incubating the biological sample with a secondary antibody or other labeling methods after detecting the target nucleic acid by in situ hybridization. In some embodiments, the target nucleic acid is RNA or DNA.

In some embodiments, the biological sample is a tissue specimen or is derived from a tissue specimen. In other embodiments, the biological sample is a blood sample or is derived from a blood sample. In yet other embodiments, the biological sample is a cytological sample or is derived from a cytological sample. In still yet other embodiments, the biological sample is cultured cells or a sample containing exosomes.

In some embodiments, the crosslinking agent in step (ii) is a fixative. In specific embodiments, the fixative is neutral buffered formalin (NBF). In one embodiment, the neutral buffered formalin is 10% neutral buffered formalin.

In some embodiments, the step of treating the biological sample with the crosslinking agent lasts for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, the step of treating the biological sample with the crosslinking agent is performed at about 4° C., room temperature, about 40° C., or about 60° C.

In some embodiments, the step of treating the biological sample with the crosslinking agent is performed at about 4° C. for about 2 hours. In other embodiments, the step of treating the biological sample with the crosslinking agent is performed at 4° C. for about 16 to about 18 hours.

In some embodiments, the step of treating the biological sample with the crosslinking agent is performed at room temperature for 15 minutes. In other embodiments, the step of treating the biological sample with the crosslinking agent is performed at room temperature for about 30 minutes. In yet other embodiments, the step of treating the biological sample with the crosslinking agent is performed at room temperature for about 60 minutes.

In some embodiments, the step of treating the biological sample with the crosslinking agent is performed at about 40° C. for about 15 minutes. In other embodiments, the step of treating the biological sample with the crosslinking agent is performed at about 40° C. for about 30 minutes. In yet other embodiments, the step of treating the biological sample with the crosslinking agent is performed at about 40° C. for about 60 minutes.

In some embodiments, the step of treating the biological sample with the crosslinking agent is performed at about 60° C. for about 15 minutes. In other embodiments, the step of treating the biological sample with the crosslinking agent is performed at about 60° C. for about 30 minutes. In yet other embodiments, the step of treating the biological sample with the crosslinking agent is performed at about 60° C. for about 60 minutes.

In another aspect, provided herein is a method for simultaneously detecting a target nucleic acid and a target protein in a biological sample, comprising: (i) incubating the biological sample with a primary antibody; (ii) treating the biological sample with a crosslinking agent; (iii) treating the biological sample with a protease; (iv) detecting the target nucleic acid by in situ hybridization; and (v) detecting the target protein by incubating the biological sample with a secondary antibody or other labeling methods.

In some embodiments, the target nucleic acid is RNA or DNA.

In some embodiments, the step of detecting the target nucleic acid by in situ hybridization comprises: (i) providing one or more target probe(s) capable of hybridizing to the target nucleic acid; (ii) providing a signal-generating complex capable of hybridizing to the one or more target probe(s), in certain embodiments, the signal-generating complex comprises a nucleic acid component capable of hybridizing to the one or more target probe(s) and a label probe; (iii) hybridizing the target nucleic acid to the one or more target probe(s); and (iv) capturing the signal-generating complex to the one or more target probe(s) and thereby capturing the signal-generating complex to the target nucleic acid. In specific embodiments, each of the one or more target probe(s) comprises a target (T) section and a label (L) section. In certain embodiments, the T section is a nucleic acid sequence complementary to a section on the target nucleic acid and the L section is a nucleic acid sequence complementary to a section on the nucleic acid component of the signal-generating complex. In certain embodiments, the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, the method further comprises providing an immunohistochemistry label capable of binding to the secondary antibody for detecting the target protein. In other embodiments, the secondary antibody is pre-labeled.

In some embodiments, the biological sample is a tissue specimen or is derived from a tissue specimen. In other embodiments, the biological sample is a blood sample or is derived from a blood sample. In yet other embodiments, the biological sample is a cytological sample or is derived from a cytological sample. In still yet other embodiments, the biological sample is cultured cells or a sample containing exosomes.

In some embodiments, the crosslinking agent is a fixative. In specific embodiments, the fixative is neutral buffered formalin. In one embodiment, the neutral buffered formalin is 10% neutral buffered formalin.

In some embodiments, the step of treating the biological sample with the crosslinking agent lasts for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, the step of treating the biological sample with the crosslinking agent is performed at about 4° C., room temperature, about 40° C., or about 60° C.

In some embodiments, the step of treating the biological sample with the crosslinking agent is performed at about 4° C. for about 2 hours. In other embodiments, the step of treating the biological sample with the crosslinking agent is performed at about 4° C. for about 16 to about 18 hours.

In some embodiments, the step of treating the biological sample with the crosslinking agent is performed at room temperature for about 15 minutes. In other embodiments, the step of treating the biological sample with the crosslinking agent is performed at room temperature for about 30 minutes. In yet other embodiments, the step of treating the biological sample with the crosslinking agent is performed at room temperature for about 60 minutes.

In some embodiments, the step of treating the biological sample with the crosslinking agent is performed at about 40° C. for about 15 minutes. In other embodiments, the step of treating the biological sample with the crosslinking agent is performed at about 40° C. for about 30 minutes. In yet other embodiments, the step of treating the biological sample with the crosslinking agent is performed at about 40° C. for about 60 minutes.

In some embodiments, the step of treating the biological sample with the crosslinking agent is performed at about 60° C. for about 15 minutes. In other embodiments, the step of treating the biological sample with the crosslinking agent is performed at about 60° C. for about 30 minutes. In yet other embodiments, the step of treating the biological sample with the crosslinking agent is performed at about 60° C. for about 60 minutes.

In some embodiments, the method is used for mapping spatial organization in a complex tissue. In certain embodiments, the complex tissue is a tumor tissue. In other embodiments, the method is used for detecting altered gene expression in the biological samples from a diseased model. In yet other embodiments, the method is used for validating novel antibodies.

In another aspect, provided herein is a kit for simultaneously detecting a target nucleic acid and a target protein in a biological sample, comprising: (i) a crosslinking agent; and (ii) an instruction indicating that the crosslinking agent is used after incubating the biological sample with a primary antibody that detects the target protein. In some embodiments, the kit further comprises a protease. In some embodiments, the kit further comprises an agent for detecting the target nucleic acid and/or an agent for detecting the target protein.

In yet another aspect, provided herein is a kit for simultaneously detecting a target nucleic acid and a target protein in a biological sample, comprising: (i) a crosslinking agent; and (ii) a protease. In some embodiments, the kit further comprises an instruction indicating that the crosslinking agent is used before the protease and the crosslinking agent is used after a primary antibody that detects the target protein. In some embodiments, the kit further comprises an agent for detecting the target nucleic acid and/or an agent for detecting the target protein. In some embodiments, the target nucleic acid is RNAor DNA.

In some embodiments, the instruction further indicates treating the biological sample with the crosslinking agent for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, the instruction further indicates treating the biological sample with the crosslinking agent at about 4° C., room temperature, about 40° C. or about 60° C.

In some embodiments, the instruction further indicates treating the biological sample with the crosslinking agent at about 4° C. for about 2 hours. In other embodiments, the instruction further indicates treating the biological sample with the crosslinking agent at about 4° C. for about 16 to about 18 hours.

In some embodiments, the instruction further indicates treating the biological sample with the crosslinking agent at room temperature for about 15 minutes. In other embodiments, the instruction further indicates treating the biological sample with the crosslinking agent at room temperature for about 30 minutes. In yet other embodiments, the instruction further indicates treating the biological sample with the crosslinking agent at room temperature for about 60 minutes.

In some embodiments, the instruction further indicates treating the biological sample with the crosslinking agent at about 40° C. for about 15 minutes. In other embodiments, the instruction further indicates treating the biological sample with the crosslinking agent at about 40° C. for about 30 minutes. In yet other embodiments, the instruction further indicates treating the biological sample with the crosslinking agent at about 40° C. for about 60 minutes.

In some embodiments, the instruction further indicates treating the biological sample with the crosslinking agent at about 60° C. for about 15 minutes. In other embodiments, the instruction further indicates treating the biological sample with the crosslinking agent at about 60° C. for about 30 minutes. In yet other embodiments, the instruction further indicates treating the biological sample with the crosslinking agent at about 60° C. for about 60 minutes.

In some embodiments, the agent for detecting the target nucleic acid comprises one or more target probe(s) capable of hybridizing to the target nucleic acid; and a signal-generating complex capable of hybridizing to the one or more target probe(s), in certain embodiments, said signal-generating complex comprises a nucleic acid component capable of hybridizing to the one or more target probe(s) and a label probe. In specific embodiments, each of the one or more target probe(s) comprises a target (T) section and a label (L) section. In certain embodiments, the T section is a nucleic acid sequence complementary to a section on the target nucleic acid and the L section is a nucleic acid sequence complementary to a section on the nucleic acid component of the signal generating complex. In certain embodiments, the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, the kit further comprises a tool for obtaining the biological sample. In specific embodiments, the biological sample is a tissue specimen or is derived from a tissue specimen. In specific embodiments, the biological sample is a blood sample or is derived from a blood sample. In specific embodiments, the biological sample is a cytological sample or is derived from a cytological sample. In specific embodiments, the biological sample is cultured cells or a sample containing exosomes.

In some embodiments, the kit is used for mapping spatial organization in a complex tissue. In specific embodiments, the complex tissue is a tumor tissue. In other embodiments, the kit is used for detecting altered gene expression in the biological samples from a diseased model. In yet other embodiments, the kit is used for validating novel antibodies.

In another aspect, provided herein is a biological sample prepared according to the method described above.

### 5. BRIEF DESCRIPTION OF THE FIGURES

FIGS. 1A-1B show schematics of sequential and ISH/IHC integrated co-detection workflow. FIG. 1A illustrates the steps in the sequential ISH-IHC workflow. FIG. 1B illustrates the steps in the ISH/IHC integrated co-detection workflow.

FIGS. 2A-2C show that crosslinking protected CD20 IHC signal against the stress of the formamide-based reagent in the hybridization buffer. FIG. 2A shows a section of Formalin Fixed Paraffin Embedded (FFPE) human tonsil tissue that was subject to IHC staining with the Leica Bond Polymer Refine Detection kit. FIG. 2B shows a section with diminished IHC signal after an exposure to hybridization buffer for 30 minutes at room temperature. FIG. 2C shows rescued IHC signal with a post-primary crosslinking despite an exposure to the hybridization buffer.

FIGS. 3A-3C show that crosslinking protected CD20 IHC signal resistance to the protease treatment. FIG. 3A shows a section of human tonsil tissue that was IHC stained to detect CD20 protein using the Leica Bond Polymer Refine Detection kit. FIG. 3B shows diminished IHC signal after exposure to protease enzyme for 15 minutes at 40° C. FIG. 3C shows rescued IHC signal with a post-primary crosslinking despite exposure to protease enzyme.

FIGS. 4A-4F show that crosslinking prior to hybridization buffer or protease treatment enhanced CD8 IHC signal. FIG. 4A shows a section of human tonsil tissue that were IHC stained to detect CDS using the Leica Bond Polymer Refine Detection kit. FIG. 4B shows diminished IHC signal after an exposure to hybridization buffer for 30 minutes at room temperature. FIG. 4C shows enhanced IHC signal with a post-primary crosslinking despite an exposure to the hybridization buffer. FIG. 4D shows a section of human tonsil that was IHC stained to detect CD8 using the Leica Bond Polymer Refine Detection kit. FIG. 4E shows diminished IHC signal after an exposure to protease enzyme for 15 minutes at 40° C. FIG. 4F shows enhanced IHC signal with a post-primary crosslinking despite an exposure to protease enzyme.

FIGS. 5A-5D show that ISH/IHC integrated co-detection workflow enabled co-detection of CD8 protein with human peptidylprolyl isomerase B (Hs-PPIB) ISH staining. FIG. 5A shows a section of FFPE human head and neck cancer sample with IHC staining against CDS with the Leica Bond Polymer Refine Detection kit. FIG. 5B shows Hs-PPIB ISH staining using the RNAscope® 2.5 LS Red kit. FIG. 5C shows ISH and IHC signals after a conventional procedure in which ISH and IHC were carried out sequentially. FIG. 5D shows enhanced ISH and IHC signals after the ISH/IHC integrated co-detection workflow.

FIGS. 6A-6E show crosslinking is key to IHC signal improvement in the ISH/IHC integrated co-detection workflow. FIG. 6A shows a section of FFPE human tonsil tissue that was subject to IHC staining against CD20 with the Leica Bond Polymer Refine Detection kit followed by Green chromogen. FIG. 6B shows a section of FFPE human tonsil tissue that was subject to ISH staining for Hs-PPIB with the RNAscope® 2.5 LS Red kit. FIG. 6C shows a section of FFPE human tonsil tissue that was subjected to RNAscope® pretreatment including a 15-minute incubation with protease at 40° C. prior to IHC staining, which resulted in decreased IHC signal. FIG. 6D shows a section of FFPE human tonsil tissue that was subjected to the ISH/IHC integrated co-detection workflow without a crosslinking step. FIG. 6E shows a section of FFPE human tonsil tissue that was subjected to the ISH/IHC integrated co-detection workflow with a crosslinking step.

FIGS. 7A-7D show crosslinking at room temperature was effective when performed for periods between 15 and 60 minutes. FIG. 7A shows a section of FFPE human stomach cancer tissue that was subject to ISH staining for detection of Hs-PPIB, and followed immediately by IHC detection of CDS, using the RNAscope® 2.5 LS Red and Leica Bond Polymer Refine Detection kits, respectively. FIG. 7B shows a section of FFPE human stomach cancer tissue that was subject to crosslinking for 15 minutes at room temperature with the ISH/IHC integrated co-detection workflow. FIG. 7C shows a section of FFPE human stomach cancer tissue that was subject to crosslinking for 30 minutes at room temperature with the ISH/IHC integrated co-detection workflow. FIG. 7D shows a section of FFPE human stomach cancer tissue that was subject to crosslinking for 60 minutes at room temperature with the ISH/IHC integrated co-detection workflow.

FIGS. 8A-8G show heated crosslinking preserved IHC signal. FIG. 8A shows a section of FFPE human stomach cancer tissue that was subject to ISH staining for detection of Hs-PPIB and followed immediately by IHC detection of CD8. FIG. 8B shows a section of FFPE human stomach cancer tissue that was subject to crosslinking for 15 minutes at 40° C. with the ISH/IHC integrated co-detection workflow. FIG. 8C shows a section of FFPE human stomach cancer tissue that was subject to crosslinking for 30 minutes at 40° C. with the ISH/IHC integrated co-detection workflow. FIG. 8D shows a section of FFPE human stomach cancer tissue that was subject to crosslinking for 60 minutes at 40° C. with the ISH/IHC integrated co-detection workflow. FIG. 8E shows a section of FFPE human stomach cancer tissue that was subject to crosslinking for 15 minutes at 60° C. with the ISH/IHC integrated co-detection workflow. FIG. 8F shows a section of human stomach cancer tissue that was subject to crosslinking for 30 minutes at 60° C. with the ISH/IHC integrated co-detection workflow. FIG. 8G shows a section of FFPE human stomach cancer tissue that was subject to crosslinking for 60 minutes at 60° C. with the ISH/IHC integrated co-detection workflow.

FIGS. 9A-9D show crosslinking at 4° C. preserved IHC signal. FIG. 9A shows a section of FFPE human stomach cancer tissue that was subject to ISH staining for detection of Hs-PPIB and followed immediately by IHC detection of CD8. FIG. 9B shows a section of FFPE human stomach cancer tissue that was subject to crosslinking for 30 minutes at room temperature with the ISH/IHC integrated co-detection workflow. FIG. 9C shows a section of FFPE human stomach cancer tissue that was subject to crosslinking for 2 hours at 4° C. with the ISH/IHC integrated co-detection workflow. FIG. 9D shows a section of FFPE human stomach cancer tissue that was subject to crosslinking overnight at 4° C. with the ISH/IHC integrated co-detection workflow.

FIGS. 10A-10B show representative workflow diagrams of the image processing methods to reduce background signals, described with reference to the images being processed (FIG. 10A) and the general steps of the method (FIG. 10B), according to one embodiment of the present disclosure.

### 6. DETAILED DESCRIPTION

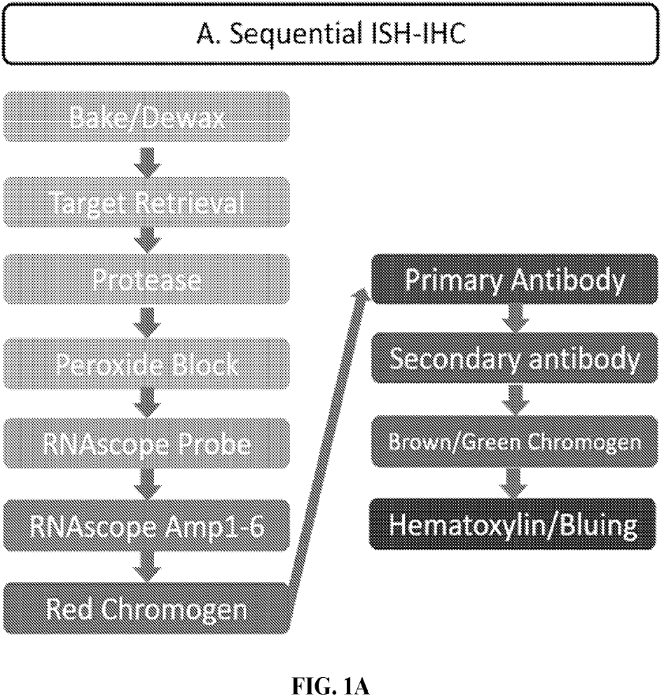

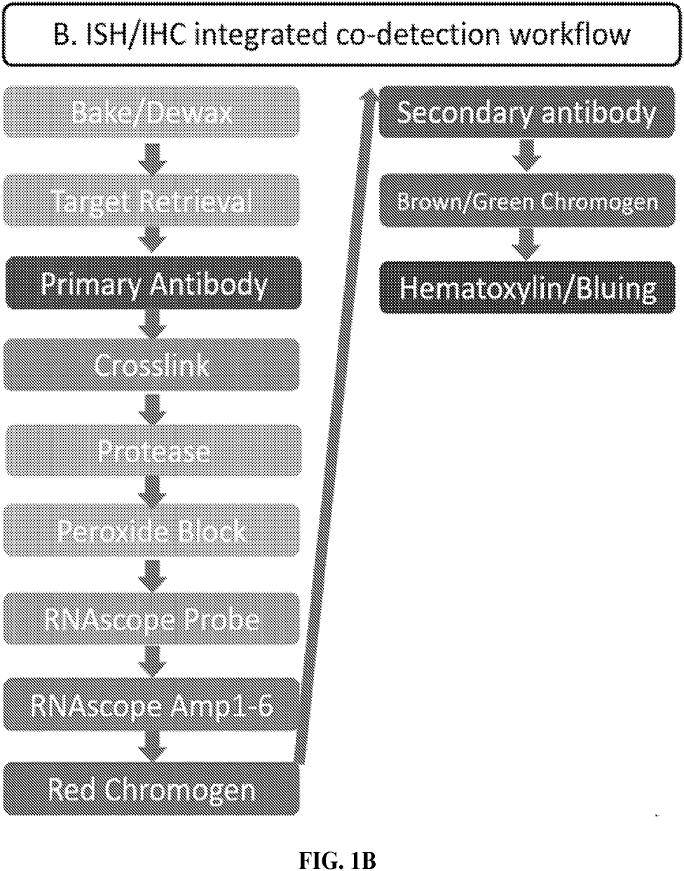

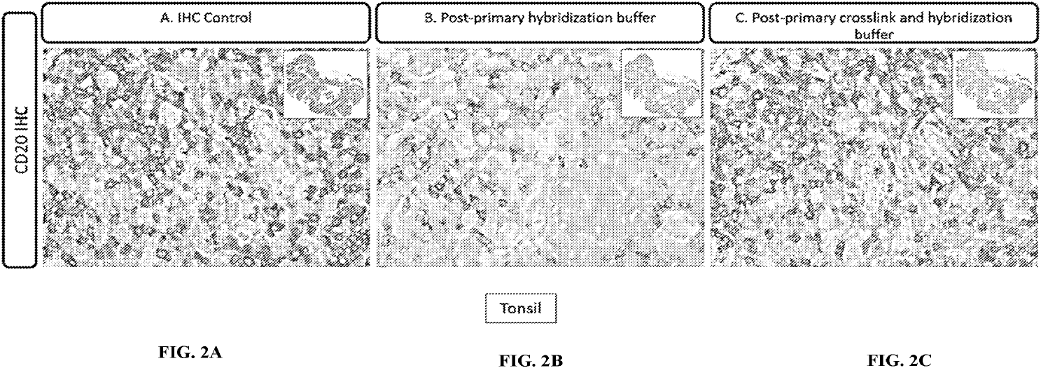

The present disclosure is based, in part, on the surprising finding that with a rearrangement of experimental procedures and the addition of a step to crosslink proteins in the sample, ISH and IHC can be effectively performed simultaneously with minimum interference. In some embodiments, to preserve and improve IHC and ISH signals, incubations of the primary antibody and the secondary antibody are separated. Furthermore, incubation with the primary antibody is followed by a step of crosslinking (see FIG. 1B), thereby protecting the binding of the primary antibody to its antigen from degradation (e.g., by protease treatment) or other interference by hybridization buffers and the high-temperature incubations commonly used in ISH (see FIGS. 2A-2C, 3A-3C and 4A-4F). This approach leads to the enhanced detection of both nucleic acids and proteins simultaneously.

### 6.1 Definitions

As used herein, the term “in situ hybridization” or “ISH” refers to a technique for localizing and visualizing specific target nucleic acids with the preservation of morphology of the source samples.

As used herein, the term “immunohistochemistry” or “IHC” generally refers to a technique for detecting proteins of interest in source samples utilizing antibodies, with the preservation of morphology of the source samples (e.g., tissue samples). As used herein, the term “immunocytochemistry” or “ICC” generally refers to a technique for detecting proteins of interest in source samples utilizing antibodies, with the preservation of morphology of the source samples (e.g., isolated or cultured intact cells, including tissue culture cell lines, either adherent or in suspension). Immunofluorescence (IF) refers to fluorescent labeling, thus it is also encompassed in the terms IHC and ICC. ICC, IHC, and IF assays can be used in conjunction with the imaging processing methods of the present disclosure, as described further herein, including facilitating quantitative and/or qualitative assessments of a target-of-interest in a sample. ICC, IHC, and IF assays can also be performed in conjunction with an in situ hybridization as part of an integrated co-detection process to detect targets-of-interest, which can also include performing the imaging processing methods of the present disclosure.

As used herein, the term “simultaneously” or “simultaneous” refers to a process of obtaining signals from different assays which are performed on the same biological sample, instead of on different samples. Therefore, it does not necessarily limit the exact timing of data acquisition or signal detection from different assays. One assay read-out could follow the other.

As used herein, the term “crosslink” refers to a process of binding two or more molecules together. The “crosslinking agent” or equivalent refers to agents containing two or more chemically reactive ends that attach themselves to the functional groups found in proteins and other molecules. Specifically, if the crosslinking agent is formaldehyde or its equivalent, a nucleophilic group on an amino acid or nucleic acid base forms a covalent bond with formaldehyde, which is stabilized in a second step that involves another functional group, often on another molecule, leading to formation of a methylene bridge. If the crosslinking agent is an oxidizing agent, it can react with the side chains of proteins and other biomolecules, allowing the formation of crosslinks that stabilize tissue structure.

As used herein, the term “primary antibody” refers to an antibody that binds directly to the antigen of interest. As used herein, the term “secondary antibody” refers to an antibody that is conjugated to a detection label. In some embodiments, the secondary antibody provided herein binds directly to the primary antibody. In other embodiments, the secondary antibody provided herein binds indirectly to the primary antibody, e.g., by binding to another antibody that recognizes the primary antibody.

As used herein, the term “fixation” or “fixing” when made in reference to fixing a biological sample in the ISH process refers to a procedure to preserve a biological sample from decay due to, e.g., autolysis or putrefaction. It terminates any ongoing biochemical reactions and may also increase the treated tissues’ mechanical strength or stability.

As used herein, the term “one or more” refers to, for example, 1 or more, 2 or more, 3 or more, 4 or more, 5 or more, 6 or more, 7 or more, 8 or more, 9 or more, 10 or more, 20 or more, 30 or more, 40 or more, 50 or more, or a greater number, if desired for a particular use.

The terms “detecting” as used herein generally refer to any form of measurement, and include determining whether an element is present or not. This term includes quantitative and/or qualitative determinations.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, cells, and exosomes isolated from a mammal. Exemplary biological samples include but are not limited to cell lysate, a cell, a cell culture, a cell line, a tissue, oral tissue, gastrointestinal tissue, an organ, an organoid, a biological fluid, a blood sample, a urine sample, a skin sample, and the like. Preferred biological samples include, but are not limited to, whole blood, partially purified blood, PBMC, tissue biopsies, and the like.

The term “probe” as used herein refers to a capture agent that is directed to a specific target mRNA sequence. Accordingly, each probe of a probe set has a respective target mRNA sequence. In some embodiments, a probe can be used individually. In other embodiments, a probe can be used among a probe set. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of′ are also provided. It is also understood that wherever embodiments are described herein with the phrase “consisting essentially of′ otherwise analogous embodiments described in terms of “consisting of are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 6.2 ISH/IHC Integrated Co-Detection

In one aspect, provided herein is a method for preparing a biological sample for simultaneously detecting a target nucleic acid and a target protein, comprising (i) incubating the biological sample with a primary antibody; (ii) treating the biological sample with a crosslinking agent after (i); and (iii) detecting the target nucleic acid by in situ hybridization after (ii).

In some embodiments, the method further comprises treating the biological sample with a protease after treating the biological sample with the crosslinking agent and before detecting the target nucleic acid by in situ hybridization. In other embodiments, the method further comprises incubating the biological sample with a secondary antibody or other labeling methods after detecting the target nucleic acid by in situ hybridization.

In some embodiments, the preparation method provided herein comprises performing the following steps sequentially: incubating the biological sample with a primary antibody; treating the biological sample with a crosslinking agent; treating the biological sample with a protease; detecting the target nucleic acid by ISH; and incubating the biological sample with a secondary antibody or other labeling methods. In some embodiments, additional steps may be included.

ISH is a powerful technique for localizing specific target nucleic acids within fixed tissues and cells, thereby, for example, obtaining temporal and spatial information about gene expression and genetic loci. A labeled probe hybridizes to a target nucleic acid sequence within a sample. This labeled probe can then be detected. ISH comprises preparing a whole-cell preparation, a paraffin-embedded tissue, a frozen tissue, or a floating section. If it is a paraffin-embedded tissue that is prepared, the next step for ISH is de-paraffinization, which is to remove paraffin, and rehydrating the sample. In some embodiments, the ISH provided herein comprises a blocking step where certain blocking agent(s) is/are applied to block certain endogenous components of the cell thus reducing assay background. For example, hydrogen peroxide is a blocking agent when horseradish peroxidase (HRP) is used as detection enzyme in the later steps. Hydrogen peroxide is added to inactivate the endogenous HRP activity in the sample, thus reducing assay background. In a specific embodiment, this blocking step is added as the first step in the pretreatment right after de-paraffinization. In some embodiments, the ISH provided herein comprises an epitope retrieval step, where certain epitope retrieval buffer(s) can be added to unmask the target nucleic acids. In some embodiments, the epitope retrieval step comprises heating the sample. In some embodiments, the epitope retrieval step comprises heating the sample to about 50° C. to about 100° C. In one embodiment, the epitope retrieval step comprises heating the sample to about 88° C. In some embodiments, the ISH provided herein comprises a protease step. Detergents (e.g., Triton X-100 or SDS) and Proteinase K can be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the target nucleic acids. Optimal concentrations and durations of treatment can be experimentally determined as is well known in the art. In some embodiments, the ISH provided herein comprises dehydrating the biological sample. In certain embodiments, the dehydration is done with ethanol of increasing concentrations, such as in the order of 70%, 95%, and 100% ethanol. In some embodiments, the ISH provided herein comprises incubating the biological sample with a hybridization buffer. In certain embodiments, the hybridization buffer is formamide-based. In some embodiments, the ISH provided herein comprises incubating the biological sample with hybridization probe(s). In some embodiments, the ISH provided herein comprises amplifying and detecting the hybridization probe(s).

Like ISH in that temporal and spatial information can be preserved, IHC is another widely used technique, but for detecting target proteins. Taking advantage of specificity of antigen-antibody binding, IHC makes it possible to visualize and document the high-resolution distribution and localization of specific target proteins within cells and within their proper histological context. IHC comprises preparing a whole-cell preparation, a paraffin-embedded tissue, a frozen tissue, or a floating section. If it is a paraffin-embedded tissue that is prepared, the next step for IHC is de-paraffinization, which is to remove paraffin. During the tissue preparation and preservation process, if a crosslinking agent, such as formalin, is used, formalin fixation may mask epitopes and result in decreased immunoreactivity (see Arnold et al., Biotech Histochem 71 :224-230(1996)). Formalin fixation is a time-dependent process in which increased fixation time results in continued formaldehyde group binding to proteins to a point of equilibrium (see Fox et al., J Histochem Cytochem 33:845-853 (1985)). Studies have shown that formalin fixation, especially if prolonged, results in decreased antigenicity (see Battifora and Kopinski, J Histochem Cytochem 34:1095-1100(1986)), which limits the use of formalin-fixed tissues for diagnostic IHC (see Ramos- Vara, Vet Pathol 42:405---426(2005), Webster et al., J Histochem Cytochem. 57(8): 753---761(2009)).

The benefits of detecting simultaneously a target nucleic acid with such as ISH and a target protein with such as IHC are enormous. For example, it increases the throughput of analysis and reduces the burden of time and cost associated with the investigation of those individual components. Furthermore, combined detection in the same sample offers researchers information which cannot be gained from staining on separate sections, such as, visualizing both a secreted protein and the origin of its cell(s), and interaction of distinct cell types. Though the steps in both techniques described above seem to be similar, it has been problematic to combine the two techniques in one sample.

In theory, an obvious option to combine ISH and IHC is to perform IHC first, then followed by performing ISH (see, e.g., Kochan et al., BioTechniques, 59(4): 209-221(2018)). Even though there are reports where immunofluorescence and RNA FISH were combined with success, these were insufficient in terms of signal intensities, staining patterns, and reagent compatibility, thus leading to artifacts and failures of the experiments. Furthermore, the IHC performed earlier may need to be RNase-free, which is not practical for larger scale or wide applications because, unless great care is taken, RNases contaminate many standard laboratory reagents.

Alternatively, detection of target proteins and target nucleic acids simultaneously on the same tissue section may be achieved by performing ISH first to preserve mRNA integrity, immediately followed by IHC (see, e.g., Stempl et al., Molecular Vision 20, 1366-1373. (2014); Grabinski et al., PLoS ONE 10(3): e0120120 (2015)). However, there are several disadvantages associated with the protocol. First of all, this approach is often not compatible with all the antibodies of interest. Second, the IHC signal that is performed later is frequently lost or greatly diminished with earlier steps of this approach. One culprit is the harsh reagents and conditions initially used for ISH, resulting in material of insufficient quality for subsequent downstream assays on the same tissue section. For example, ISH pretreatment uses proteases to assist with reagent penetration and target accessibility. This protease step can potentially damage epitopes, thereby disrupting antibody-antigen binding and consequently reducing the IHC signal. In addition, formamide-based buffers and high-temperature incubation commonly used in ISH can disrupt non-covalent antibody-antigen binding, thereby reducing IHC signal.

The methods provided herein overcome the above-mentioned challenges by modifying the conventional protocol by, for example, stabilizing antigen-antibody binding. The stabilization is accomplished by, for example, incubating the sample with the primary antibody first and fixing samples with 10% NBF prior to ISH detection, which is then followed by the remaining IHC staining.

In some specific embodiments, the methods provided herein comprise crosslinking the samples after primary antibody binding, the crosslinking is followed by protease treatment and ISH and then subsequently followed by the remaining IHC staining steps. The method has the capacity to both protect the epitope from protease activity and to stabilize the antigen-antibody binding through subsequent steps involving hybridization buffer-dependent amplification. The method offers a greater flexibility in co-detection of proteins and nucleic acids, maintains high-resolution detection of nucleic acids, and stabilizes the IHC signal of antibodies previously incompatible with the conventional co-detection.

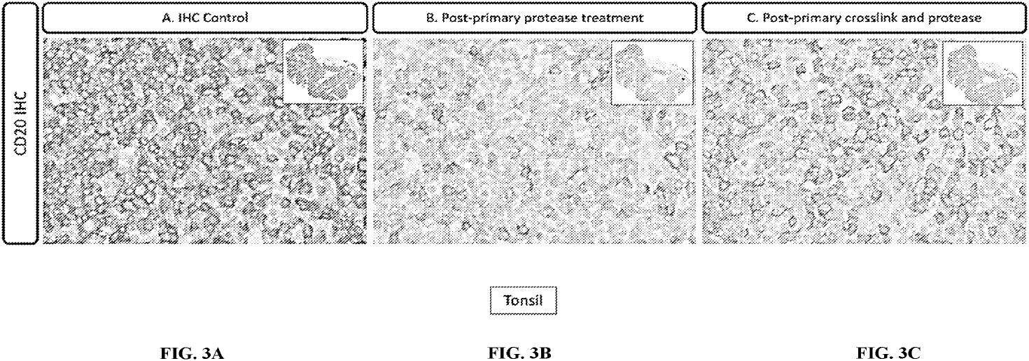

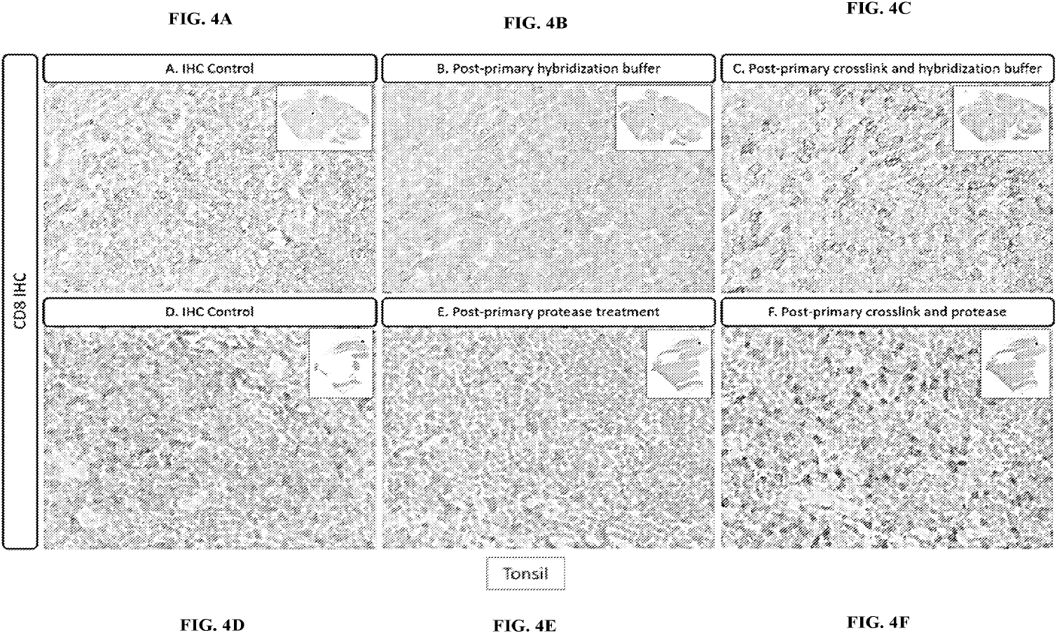

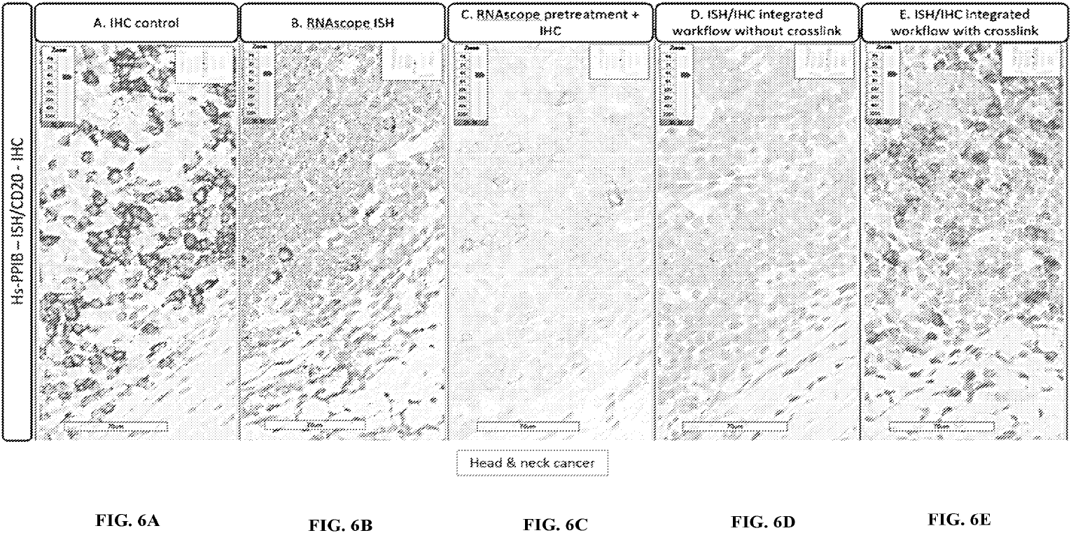

As illustrated in Sections 7.2 and 7.3 below, the method provided herein enables the detection of antibodies that were deemed incompatible with the conventional co-detection of ISH and IHC, such as CD20. Conventionally, if ISH is performed first to preserve signal from the target nucleic acids, IHC signal may be compromised. For example, the same sample has been processed by ISH procedures, such as protease treatment and others, the epitope may be negatively affected. Therefore, incubating the sample with antibodies before the potential problematic ISH procedures is beneficial. Furthermore, as illustrated in FIGS. 6C-6E, a step of crosslinking following the incubation of primary antibodies is added for the ISH and IHC integration. Crosslinking improved antigen-antibody binding, and allowed for detection and visualization of protein epitopes not previously compatible with ISH conditions. In addition, the crosslinking step provided herein is shown to improve IHC signal by protecting the antibody-antigen binding in the samples from protease treatment and the incubation of hybridization buffers (see FIGS. 3A-3C and FIGS. 4A-3C).

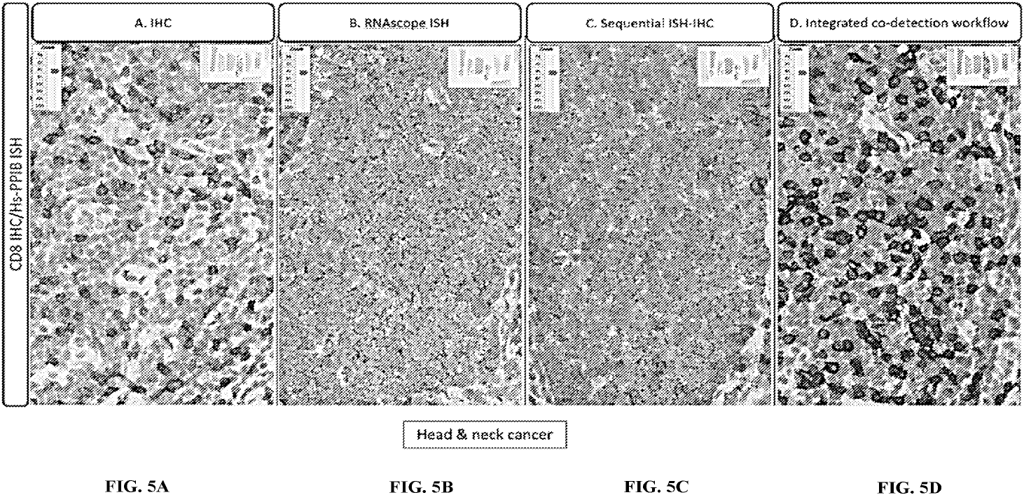

Furthermore, the methods provided herein demonstrated a further boosting effect for some antibodies of interest. For example, crosslinking prior to the incubation of hybridization buffers and/or protease treatment improved IHC signal for a CD8 antibody when compared to not only the conventional ISH-IHC protocol, but also the standard IHC protocol alone (see FIGS. 4A-4F and 5A-5D). A similar boost in IHC detection applies to additional antibodies. In some embodiments, in an effort to detect target proteins alone, similar procedures such as an extra crosslinking step after the incubation of the primary antibodies, may be employed to enhance IHC signal. In other embodiments, in an effort to detect target proteins alone, protease treatment may be employed to enhance IHC signal. In other embodiments, in an effort to detect target proteins alone, procedures, such as an extra crosslinking step after the incubation of the primary antibodies and experimental steps that mimic partial or all procedures of ISH, may be employed to enhance IHC signal.

Before hybridization with ISH probe(s), several procedures of pretreatment may be needed. For example, an ISH blocking is when certain blocking agent(s) is/are applied to block certain endogenous components of the cell thus reducing assay background. For example, hydrogen peroxide is a blocking agent when horseradish peroxidase (HRP) is used as detection enzyme in the later steps. Hydrogen peroxide is added to inactivate the endogenous HRP activity in the sample, thus reducing assay background. In a specific embodiment, this ISH blocking is added as the first step in the pretreatment right after de-paraffinization. In some embodiments, the pretreatment step comprises a target retrieval step, where certain target retrieval buffer(s) can be added to unmask the target nucleic acid. In some embodiments, the epitope retrieval step comprises heating the sample. In some embodiments, the target retrieval step comprises heating the sample to about 50° C. to about 100° C. In one embodiment, the target retrieval step comprises heating the sample to about 88° C. In some embodiments, the pretreatment step comprises protease treatment, which is used to digest proteins that surround the target nucleic acids. Optimal concentrations and durations of treatment can be experimentally determined as is well known in the art. In certain embodiments, the protease treatment is performed with trypsin. In certain embodiments, the protease treatment is performed with proteinase K. In certain embodiments, the protease treatment is performed with pepsin. In certain embodiments, the protease treatment is performed with pronase. In certain embodiments, the protease treatment is performed with endoproteinase AspN. In certain embodiments, the protease treatment is performed with endoproteinase GluC.

In some embodiments, the method provided herein, for preparing a biological sample for simultaneously detecting a target nucleic acid and a target protein, comprises incubating the biological sample with a primary antibody, followed by first treating the biological sample with a crosslinking agent; next detecting the target nucleic acid by ISH; and then the remaining steps for IHC. In other embodiments, the method provided herein comprises incubating the biological sample with a primary antibody, followed by first treating the biological sample with a crosslinking agent, next protease treatment, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next protease treatment, next ISH blocking, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next ISH blocking, next protease treatment, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next ISH blocking, next detecting the target nucleic acid by ISH, and then remaining steps for IHC, as described in the immediately preceding paragraph. In other embodiments, the method provided herein comprises target retrieval, next incubating the biological sample with a primary antibody, and followed by first treating the biological sample with a crosslinking agent, next detecting the target nucleic acid by ISH, and then the remaining steps for IHC, as described in the immediately preceding paragraph. In other embodiments, the method provided herein comprises target retrieval, next incubating the biological sample with a primary antibody, and followed by first treating the biological sample with a crosslinking agent, then protease treatment, next detecting the target nucleic acid by ISH, and then the remaining steps for IHC, as described in the immediately preceding paragraph. In other embodiments, the method provided herein comprises target retrieval, next incubating the biological sample with a primary antibody, and followed by first treating the biological sample with a crosslinking agent, next protease treatment, next ISH blocking, next detecting the target nucleic acid by ISH, and then the remaining steps for IHC, as described in the immediately preceding paragraph. In other embodiments, the method provided herein comprises target retrieval, next incubating the biological sample with a primary antibody, and followed by first treating the biological sample with a crosslinking agent, next ISH blocking, next protease treatment, next detecting the target nucleic acid by ISH, and then the remaining steps for IHC, as described in the immediately preceding paragraph. In other embodiments, the method provided herein comprises incubating target retrieval, next the biological sample with a primary antibody, and followed by first treating the biological sample with a crosslinking agent, next ISH blocking, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next target retrieval, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next target retrieval, next protease treatment, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next target retrieval, next protease treatment, next ISH blocking, next detecting the target nucleic acid by ISH, and then the remaining steps for IHC, as described in the immediately preceding paragraph. In other embodiments, the method provided herein comprises incubating the biological sample with a primary antibody, followed first treating the biological sample with a crosslinking agent, next target retrieval, next ISH blocking, next protease treatment, next detecting the target nucleic acid by ISH, and then the remaining steps for IHC, as described in the immediately preceding paragraph. In other embodiments, the method provided herein comprises incubating the biological sample with a primary antibody, followed by first treating the biological sample with a crosslinking agent, next target retrieval, next ISH blocking, next detecting the target nucleic acid by ISH, and then the remaining steps for IHC, as described in the immediately preceding paragraph. In other embodiments, the method provided herein comprises utilizing more than two antibodies, the second or other follow-on antibodies are applied after the primary antibody, and the secondary antibody or other detection method is applied after ISH.

In some embodiments, the method provided herein comprises treating the biological sample with a crosslinking agent. In certain embodiments, the crosslinking agent is a fixative. In specific embodiments, the crosslinking agent is formaldehyde. In specific embodiments, the crosslinking agent is glutaraldehyde. In specific embodiments, the crosslinking agent is acrolein. In specific embodiments, the crosslinking agent is osmium tetroxide. In specific embodiments, the crosslinking agent is a type of permanganate fixative. In one embodiment, the crosslinking agent is potassium permanganate. In specific embodiments, the crosslinking agent is a type of dichromate fixative. In one embodiment, the crosslinking agent is potassium dichromate. In specific embodiments, the crosslinking agent is chromic acid.

In some embodiments, the method provided herein comprises treating the biological sample with a crosslinking agent that is a mixture of crosslinking fixatives. In some embodiments, the crosslinking agent is a mixture solution of two or more fixatives, selected from a list of formaldehyde, glutaraldehyde, acrolein, osmium tetroxide, permanganate fixative, dichromate fixative, and chromic acid. In one embodiment, the crosslinking agent is Bouin’s fixative, which is a solution of picric acid, formaldehyde, and acetic acid. In one embodiment, the crosslinking agent is a mixture of formaldehyde and glutaraldehyde. In one embodiment, the crosslinking agent is FAA, which is a solution of ethanol, acetic acid, and formaldehyde. In one embodiment, the crosslinking agent is periodate-lysine-paraformaldehyde (PLP), which is a solution of paraformaldehyde, L-lysine, and INaO4. In one embodiment, the crosslinking agent is phosphate buffered formalin (PBF). In one embodiment, the crosslinking agent is formal calcium, which is a solution of formaldehyde and calcium chloride. In one embodiment, the crosslinking agent is formal saline, which is a solution of formaldehyde and sodium chloride. In one embodiment, the crosslinking agent is zinc formalin, which is a solution of formaldehyde and zinc sulphate. In one embodiment, the crosslinking agent is Helly’s fixative, which is a solution of formaldehyde, potassium dichromate, sodium sulphate, and mercuric chloride. In one embodiment, the crosslinking agent is Hollande’s fixative, which is a solution of formaldehyde, copper acetate, picric acid, and acetic acid. In one embodiment, the crosslinking agent is Gendre’s solution, which is a solution of formaldehyde, ethanol, picric acid, and acetic acid glacial. In one embodiment, the crosslinking agent is alcoholic formalin, which is a solution of formaldehyde, ethanol, and calcium acetate. In one embodiment, the crosslinking agent is formol acetic alcohol, which is a solution of formaldehyde, acetic acid glacial, and ethanol. In one embodiment, the crosslinking agent is a mixture of fixatives, wherein at least one fixative of the mixture is formaldehyde or glutaraldehyde. In one embodiment, the crosslinking agent is fixatives that are not used at the same time but separately or consecutively, wherein at least one fixative is formaldehyde or glutaraldehyde.

In some embodiments, the method provided herein comprises treating the biological sample with two or more crosslinking agents, which are not applied at the same time but separately or consecutively and are selected from a list of formaldehyde, glutaraldehyde, acrolein, osmium tetroxide, permanganate fixative, dichromate fixative, and chromic acid.

In some embodiments, the method provided herein comprises treating the biological sample with a crosslinking agent at a temperature of about 0° C. to about 100° C. In some embodiments, the method provided herein comprises treating the biological sample with a crosslinking agent at a temperature of about 1° C. to about 90° C. In some embodiments, the method provided herein comprises treating the biological sample with a crosslinking agent at a temperature of about 2° C. to about 80° C. In some embodiments, the method provided herein comprises treating the biological sample with a crosslinking agent at a temperature of about 3° C. to about 70° C. In some embodiments, the method provided herein comprises treating the biological sample with a crosslinking agent at a temperature of about 4° C. to about 60° C.

In some embodiments, the method provided herein comprises treating the biological sample with about 1 % to about 20% neutral buffered formalin (NBF) at a temperature of about 0° C. to about 100° C. In some embodiments, the method provided herein comprises treating the biological sample with about 1% to about 20% NBF at a temperature of about 1° C. to about 90° C. In some embodiments, the method provided herein comprises treating the biological sample with about 1% to about 20% NBF at a temperature of about 2° C. to about 80° C. In some embodiments, the method provided herein comprises treating the biological sample with about 1% to about 20% NBF at a temperature of about 3° C. to about 70° C. In some embodiments, the method provided herein comprises treating the biological sample with about 1°% to about 20% NBF at a temperature of about 4° C. to about 60° C.

In some embodiments, the method provided herein comprises treating the biological sample with about 10% NBF at a temperature of about 0° C. to about 100° C. In some embodiments, the method provided herein comprises treating the biological sample with about 10% NBF at a temperature of about 1° C. to about 90° C. In some embodiments, the method provided herein comprises treating the biological sample with 10% NBF at a temperature of about 2° C. to about 80° C. In some embodiments, the method provided herein comprises treating the biological sample with about 10% NBF at a temperature of about 3° C. to about 70° C. In some embodiments, the method provided herein comprises treating the biological sample with about 10% NBF at a temperature of about 4° C. to about 60° C.

In some embodiments, the method provided herein comprises treating the biological sample with a crosslinking agent at a temperature of about 1° C. In some embodiments, the method provided herein comprises treating the biological sample with a crosslinking agent at a temperature of about 2° C. In some embodiments, the method provided herein comprises treating the biological sample with a crosslinking agent at a temperature of about 3° C. In some embodiments, the method provided herein comprises treating the biological sample with a crosslinking agent at a temperature of about 4° C. In some embodiments, the method provided herein comprises treating the biological sample with a crosslinking agent at a temperature of about 5° C. In some embodiments, the method provided herein comprises treating the biological sample with a crosslinking agent at a temperature of about 6° C. In some embodiments, the method provided herein comprises treating the biological sample with a crosslinking agent at a temperature of about 7° C. In some embodiments, the method provided herein comprises treating the biological sample with a crosslinking agent at a temperature of about 8° C. In some embodiments, the method provided herein comprises treating the biological sample with a crosslinking agent at a temperature of about 9° C. In some embodiments, the method provided herein comprises treating the biological sample with a crosslinking agent at a temperature of about 10° C. In some embodiments, the method provided herein comprises treating the biological sample with a crosslinking agent at a temperature of about 11° C. In some embodiments, the method provided herein comprises treating the biological sample with a crosslinking agent at a temperature of about 12° C. In some embodiments, the method provided herein comprises treating the biological sample with a crosslinking agent at a temperature of about 13° C. In some embodiments, the method provided herein comprises treating the biological sample with a crosslinking agent at a temperature of about 14° C. In some embodiments, the method provided herein comprises treating the biological sample with a crosslinking agent at a temperature of about 15° C. In some embodiments, the method provided herein comprises treating the biological sample with a crosslinking agent at a temperature of about 16° C. In some embodiments, the method provided herein comprises treating the biological sample with a crosslinking agent at a temperature of about 17° C. In some embodiments, the method provided herein comprises treating the biological sample with a crosslinking agent at a temperature of about 18° C. In some embodiments, the method provided herein comprises treating the biological sample with a crosslinking agent at a temperature of about 19° C. In some embodiments, the method provided herein comprises treating the biological sample with a crosslinking agent at a temperature of about 20° C. In some embodiments, the method provided herein comprises treating the biological sample with a crosslinking agent at a temperature of about 21° C. In some embodiments, the method provided herein comprises treating the biological sample with a crosslinking agent at a temperature of about 22° C. In some embodiments, the method provided herein comprises treating the biological sample with a crosslinking agent at a temperature of about 23° C. In some embodiments, the method provided herein comprises treating the biological sample with a crosslinking agent at a temperature of about 24° C. In some embodiments, the method provided herein comprises treating the biological sample with a crosslinking agent at a temperature of about 25° C. In some embodiments, the method provided herein comprises treating the biological sample with a crosslinking agent at a temperature of about 26° C. In some embodiments, the method provided herein comprises treating the biological sample with a crosslinking agent at a temperature of about 27° C. In some embodiments, the method provided herein comprises treating the biological sample with a crosslinking agent at a temperature of about 28° C. In some embodiments, the method provided herein comprises treating the biological sample with a crosslinking agent at a temperature of about 29° C. In some embodiments, the method provided herein comprises treating the biological sample with a crosslinking agent at a temperature of about 30° C. In some embodiments, the method provided herein comprises treating the biological sample with a crosslinking agent at a temperature of about 35° C. In some embodiments, the method provided herein comprises treating the biological sample with a crosslinking agent at a temperature of about 40° C. In some embodiments, the method provided herein comprises treating the biological sample with a crosslinking agent at a temperature of about 45° C. In some embodiments, the method provided herein comprises treating the biological sample with a crosslinking agent at a temperature of about 50° C. In some embodiments, the method provided herein comprises treating the biological sample with a crosslinking agent at a temperature of about 55° C. In some embodiments, the method provided herein comprises treating the biological sample with a crosslinking agent at a temperature of about 60° C. In some embodiments, the method provided herein comprises treating the biological sample with a crosslinking agent at a temperature of about 65° C. In some embodiments, the method provided herein comprises treating the biological sample with a crosslinking agent at a temperature of about 70° C. In some embodiments, the method provided herein comprises treating the biological sample with a crosslinking agent at a temperature of about 75° C. In some embodiments, the method provided herein comprises treating the biological sample with a crosslinking agent at a temperature of about 80° C. In some embodiments, the method provided herein comprises treating the biological sample with a crosslinking agent at a temperature of about 85° C. In some embodiments, the method provided herein comprises treating the biological sample with a crosslinking agent at a temperature of about 90° C. In some embodiments, the method provided herein comprises treating the biological sample with a crosslinking agent at a temperature of about 95° C. In some embodiments, the method provided herein comprises treating the biological sample with a crosslinking agent at a temperature of about 100° C.

In some embodiments, the method provided herein comprises treating the biological sample with 1 % to 20% NBF at a temperature of about 1° C. In some embodiments, the method provided herein comprises treating the biological sample with 1% to 20% NBF at a temperature of about 2° C. In some embodiments, the method provided herein comprises treating the biological sample with 1% to 20% NBF at a temperature of about 3° C. In some embodiments, the method provided herein comprises treating the biological sample with 1 % to 20% NBF at a temperature of about 4° C. In some embodiments, the method provided herein comprises treating the biological sample with 1% to 20% NBF at a temperature of about 5° C. In some embodiments, the method provided herein comprises treating the biological sample with 1% to 20% NBF at a temperature of about 6° C. In some embodiments, the method provided herein comprises treating the biological sample with 1% to 20% NBF at a temperature of about 7° C. In some embodiments, the method provided herein comprises treating the biological sample with 1% to 20% NBF at a temperature of about 8° C. In some embodiments, the method provided herein comprises treating the biological sample with 1% to 20% NBF at a temperature of about 9° C. In some embodiments, the method provided herein comprises treating the biological sample with 1% to 20% NBF at a temperature of about 10° C. In some embodiments, the method provided herein comprises treating the biological sample with 1% to 20% NBF at a temperature of about 11° C. In some embodiments, the method provided herein comprises treating the biological sample with 1% to 20% NBF at a temperature of about 12° C. In some embodiments, the method provided herein comprises treating the biological sample with 1% to 20% NBF at a temperature of about 13° C. In some embodiments, the method provided herein comprises treating the biological sample with 1% to 20% NBF at a temperature of about 14° C. In some embodiments, the method provided herein comprises treating the biological sample with 1% to 20% NBF at a temperature of about 15° C. In some embodiments, the method provided herein comprises treating the biological sample with 1% to 20% NBF at a temperature of about 16° C. In some embodiments, the method provided herein comprises treating the biological sample with 1% to 20% NBF at a temperature of about 17° C. In some embodiments, the method provided herein comprises treating the biological sample with 1% to 20% NBF at a temperature of about 18° C. In some embodiments, the method provided herein comprises treating the biological sample with 1% to 20% NBF at a temperature of about 19° C. In some embodiments, the method provided herein comprises treating the biological sample with 1% to 20% NBF at a temperature of about 20° C. In some embodiments, the method provided herein comprises treating the biological sample with 1% to 20%; NBF at a temperature of about 21° C. In some embodiments, the method provided herein comprises treating the biological sample with 1% to 20% NBF at a temperature of about 22° C. In some embodiments, the method provided herein comprises treating the biological sample with 1% to 20% NBF at a temperature of about 23° C. In some embodiments, the method provided herein comprises treating the biological sample with 1% to 20% NBF at a temperature of about 24° C. In some embodiments, the method provided herein comprises treating the biological sample with 1% to 20% NBF at a temperature of about 25° C. In some embodiments, the method provided herein comprises treating the biological sample with 1% to 20% NBF at a temperature of about 26° C. In some embodiments, the method provided herein comprises treating the biological sample with 1% to 20% NBF at a temperature of about 27° C. In some embodiments, the method provided herein comprises treating the biological sample with 1% to 20% NBF at a temperature of about 28° C. In some embodiments, the method provided herein comprises treating the biological sample with 1% to 20% NBF at a temperature of about 29° C. In some embodiments, the method provided herein comprises treating the biological sample with 1% to 20% NBF at a temperature of about 30° C. In some embodiments, the method provided herein comprises treating the biological sample with 1% to 20% NBF at a temperature of about 35° C. In some embodiments, the method provided herein comprises treating the biological sample with 1% to 20% NBF at a temperature of about 40° C. In some embodiments, the method provided herein comprises treating the biological sample with 1% to 20% NBF at a temperature of about 45° C. In some embodiments, the method provided herein comprises treating the biological sample with 1% to 20% NBF at a temperature of about 50° C. In some embodiments, the method provided herein comprises treating the biological sample with 1% to 20%; NBF at a temperature of about 55° C. In some embodiments, the method provided herein comprises treating the biological sample with 1% to 20% NBF at a temperature of about 60° C. In some embodiments, the method provided herein comprises treating the biological sample with 1% to 20% NBF at a temperature of about 65° C. In some embodiments, the method provided herein comprises treating the biological sample with 1% to 20% NBF at a temperature of about 70° C. In some embodiments, the method provided herein comprises treating the biological sample with 1% to 20% NBF at a temperature of about 75° C. In some embodiments, the method provided herein comprises treating the biological sample with 1% to 20% NBF at a temperature of about 80° C. In some embodiments, the method provided herein comprises treating the biological sample with 1% to 20% NBF at a temperature of about 85° C. In some embodiments, the method provided herein comprises treating the biological sample with 1% to 20% NBF at a temperature of about 90° C. In some embodiments, the method provided herein comprises treating the biological sample with 1% to 20% NBF at a temperature of about 95° C. In some embodiments, the method provided herein comprises treating the biological sample with 1% to 20% NBF at a temperature of about 100° C.

In some embodiments, the method provided herein comprises treating the biological sample with 10% NBF at a temperature of about 1° C. In some embodiments, the method provided herein comprises treating the biological sample with 10% NBF at a temperature of about 2° C. In some embodiments, the method provided herein comprises treating the biological sample with 10% NBF at a temperature of about 3° C. In some embodiments, the method provided herein comprises treating the biological sample with 10% NBF at a temperature of about 4° C. In some embodiments, the method provided herein comprises treating the biological sample with 1 0% NBF at a temperature of about 5° C. In some embodiments, the method provided herein comprises treating the biological sample with 10% NBF at a temperature of about 6° C. In some embodiments, the method provided herein comprises treating the biological sample with 10% NBF at a temperature of about 7° C. In some embodiments, the method provided herein comprises treating the biological sample with 1 0% NBF at a temperature of about 8° C. In some embodiments, the method provided herein comprises treating the biological sample with 10% NBF at a temperature of about 9° C. In some embodiments, the method provided herein comprises treating the biological sample with 10% NBF at a temperature of about 10° C. In some embodiments, the method provided herein comprises treating the biological sample with 10% NBF at a temperature of about 11° C. In some embodiments, the method provided herein comprises treating the biological sample with 10% NBF at a temperature of about 12° C. In some embodiments, the method provided herein comprises treating the biological sample with 10% NBF at a temperature of about 13° C. In some embodiments, the method provided herein comprises treating the biological sample with 10% NBF at a temperature of about 14° C. In some embodiments, the method provided herein comprises treating the biological sample with 10% NBF at a temperature of about 15° C. In some embodiments, the method provided herein comprises treating the biological sample with 10% NBF at a temperature of about 16° C. In some embodiments, the method provided herein comprises treating the biological sample with 10% NBF at a temperature of about 17° C. In some embodiments, the method provided herein comprises treating the biological sample with 10% NBF at a temperature of about 18° C. In some embodiments, the method provided herein comprises treating the biological sample with 10% NBF at a temperature of about 19° C. In some embodiments, the method provided herein comprises treating the biological sample with 10% NBF at a temperature of about 20° C. In some embodiments, the method provided herein comprises treating the biological sample with 1 0% NBF at a temperature of about 21° C. In some embodiments, the method provided herein comprises treating the biological sample with 10% NBF at a temperature of about 22° C. In some embodiments, the method provided herein comprises treating the biological sample with 10% NBF at a temperature of about 23° C. In some embodiments, the method provided herein comprises treating the biological sample with 10% NBF at a temperature of about 24° C. In some embodiments, the method provided herein comprises treating the biological sample with 10% NBF at a temperature of about 25° C. In some embodiments, the method provided herein comprises treating the biological sample with 10% NBF at a temperature of about 26° C. In some embodiments, the method provided herein comprises treating the biological sample with 1 0% NBF at a temperature of about 27° C. In some embodiments, the method provided herein comprises treating the biological sample with 10% NBF at a temperature of about 28° C. In some embodiments, the method provided herein comprises treating the biological sample with 10% NBF at a temperature of about 29° C. In some embodiments, the method provided herein comprises treating the biological sample with 10% NBF at a temperature of about 30° C. In some embodiments, the method provided herein comprises treating the biological sample with 10% NBF at a temperature of about 35° C. In some embodiments, the method provided herein comprises treating the biological sample with 10% NBF at a temperature of about 40° C. In some embodiments, the method provided herein comprises treating the biological sample with 1 0% NBF at a temperature of about 45° C. In some embodiments, the method provided herein comprises treating the biological sample with 10% NBF at a temperature of about 50° C. In some embodiments, the method provided herein comprises treating the biological sample with 10% NBF at a temperature of about 55° C. In some embodiments, the method provided herein comprises treating the biological sample with 10% NBF at a temperature of about 60° C. In some embodiments, the method provided herein comprises treating the biological sample with 10% NBF at a temperature of about 65° C. In some embodiments, the method provided herein comprises treating the biological sample with 10% NBF at a temperature of about 70° C. In some embodiments, the method provided herein comprises treating the biological sample with 10% NBF at a temperature of about 75° C. In some embodiments, the method provided herein comprises treating the biological sample with 10% NBF at a temperature of about 80° C. In some embodiments, the method provided herein comprises treating the biological sample with 10% NBF at a temperature of about 85° C. In some embodiments, the method provided herein comprises treating the biological sample with 1 0% NBF at a temperature of about 90° C. In some embodiments, the method provided herein comprises treating the biological sample with 10% NBF at a temperature of about 95° C. In some embodiments, the method provided herein comprises treating the biological sample with 10% NBF with 10% NBF at a temperature of about 100° C.

In some embodiments, the method provided herein comprises treating the biological sample with a crosslinking agent for about 0.1 hour to about 48 hours. In some embodiments, the method provided herein comprises treating the biological sample with a crosslinking agent for about 0.1 hour to about 36 hours. In some embodiments, the method provided herein comprises treating the biological sample with a crosslinking agent for about 0.1 hour to about 24 hours. In some embodiments, the method provided herein comprises treating the biological sample with a crosslinking agent for about 0.2 hour to about 22 hours. In some embodiments, the method provided herein comprises treating the biological sample with a crosslinking agent for about 0.25 hour to about 20 hours. In some embodiments, the method provided herein comprises treating the biological sample with a crosslinking agent for about 0.25 hour to about 18 hours.

In some embodiments, the method provided herein comprises treating the biological sample with 1% to 20% NBF for about 0.1 hour to about 48 hours. In some embodiments, the method provided herein comprises treating the biological sample with 1% to 20% NBF for about 0.1 hour to about 36 hours. In some embodiments, the method provided herein comprises treating the biological sample with 1% to 20% NBF for about 0.1 hour to about 24 hours. In some embodiments, the method provided herein comprises treating the biological sample with 1% to 20% NBF for about 0.2 hour to about 22 hours. In some embodiments, the method provided herein comprises treating the biological sample with 1% to 20% NBF for about 0.25 hour to about 20 hours. In some embodiments, the method provided herein comprises treating the biological sample with 1% to 20% NBF for about 0.25 hour to about 18 hours.

In some embodiments, the method provided herein comprises treating the biological sample with 10% NBF for about 0.1 hour to about 48 hours. In some embodiments, the method provided herein comprises treating the biological sample with 10% NBF for about 0.1 hour to about 36 hours. In some embodiments, the method provided herein comprises treating the biological sample with 10% NBF for about 0.1 hour to about 24 hours. In some embodiments, the method provided herein comprises treating the biological sample with 10% NBF for about 0.2 hour to about 22 hours. In some embodiments, the method provided herein comprises treating the biological sample with 10% NBF for about 0.25 hour to about 20 hours. In some embodiments, the method provided herein comprises treating the biological sample with 10% NBF for about 0.25 hour to about 18 hours.

In some embodiments, the method provided herein comprises treating the biological sample with a crosslinking agent for about 0.1 hour. In some embodiments, the method provided herein comprises treating the biological sample with a crosslinking agent for about 0.25 hour. In some embodiments, the method provided herein comprises treating the biological sample with a crosslinking agent for about 0.5 hour. In some embodiments, the method provided herein comprises treating the biological sample with a crosslinking agent for about 0.75 hour. In some embodiments, the method provided herein comprises treating the biological sample with a crosslinking agent for about 1 hour. In some embodiments, the method provided herein comprises treating the biological sample with a crosslinking agent for about 2 hours. In some embodiments, the method provided herein comprises treating the biological sample with a crosslinking agent for about 3 hours. In some embodiments, the method provided herein comprises treating the biological sample with a crosslinking agent for about 4 hours. In some embodiments, the method provided herein comprises treating the biological sample with a crosslinking agent for about 5 hours. In some embodiments, the method provided herein comprises treating the biological sample with a crosslinking agent for about 6 hours. In some embodiments, the method provided herein comprises treating the biological sample with a crosslinking agent for about 7 hours. In some embodiments, the method provided herein comprises treating the biological sample with a crosslinking agent for about 8 hours. In some embodiments, the method provided herein comprises treating the biological sample with a crosslinking agent for about 9 hours. In some embodiments, the method provided herein comprises treating the biological sample with a crosslinking agent for about 10 hours. In some embodiments, the method provided herein comprises treating the biological sample with a crosslinking agent for about 11 hours. In some embodiments, the method provided herein comprises treating the biological sample with a crosslinking agent for about 12 hours. In some embodiments, the method provided herein comprises treating the biological sample with a crosslinking agent for about 14 hours. In some embodiments, the method provided herein comprises treating the biological sample with a crosslinking agent for about 18 hours. In some embodiments, the method provided herein comprises treating the biological sample with a crosslinking agent for about 20 hours. In some embodiments, the method provided herein comprises treating the biological sample with a crosslinking agent for about 24 hours. In some embodiments, the method provided herein comprises treating the biological sample with a crosslinking agent for about 36 hours. In some embodiments, the method provided herein comprises treating the biological sample with a crosslinking agent for about 48 hours.

In some embodiments, the method provided herein comprises treating the biological sample with 1% to 20% NBF for about 0.1 hour. In some embodiments, the method provided herein comprises treating the biological sample with 1% to 20% NBF for about 0.25 hour. In some embodiments, the method provided herein comprises treating the biological sample with 10% for about 0.5 hour. In some embodiments, the method provided herein comprises treating the biological sample with 1% to 20% NBF for about 0.75 hour. In some embodiments, the method provided herein comprises treating the biological sample with 1% to 20% NBF for about 1 hour. In some embodiments, the method provided herein comprises treating the biological sample with 1% to 20% NBF for about 2 hours. In some embodiments, the method provided herein comprises treating the biological sample with 1% to 20% NBF for about 3 hours. In some embodiments, the method provided herein comprises treating the biological sample with 1% to 20% NBF for about 4 hours. In some embodiments, the method provided herein comprises treating the biological sample with 1% to 20% NBF for about 5 hours. In some embodiments, the method provided herein comprises treating the biological sample with 1% to 20% NBF for about 6 hours. In some embodiments, the method provided herein comprises treating the biological sample with 1% to 20% NBF for about 7 hours. In some embodiments, the method provided herein comprises treating the biological sample with 1% to 20% NBF for about 8 hours. In some embodiments, the method provided herein comprises treating the biological sample with 1% to 20% NBF for about 9 hours. In some embodiments, the method provided herein comprises treating the biological sample with 1% to 20% NBF for about 10 hours. In some embodiments, the method provided herein comprises treating the biological sample with 1% to 20% NBF for about 11 hours. In some embodiments, the method provided herein comprises treating the biological sample with 1% to 20% NBF for about 12 hours. In some embodiments, the method provided herein comprises treating the biological sample with 1% to 20% NBF for about 14 hours. In some embodiments, the method provided herein comprises treating the biological sample with 1% to 20% NBF for about 18 hours. In some embodiments, the method provided herein comprises treating the biological sample with 1% to 20% NBF for about 20 hours. In some embodiments, the method provided herein comprises treating the biological sample with 1% to 20% NBF for about 24 hours. In some embodiments, the method provided herein comprises treating the biological sample with 1% to 20% NBF for about 36 hours. In some embodiments, the method provided herein comprises treating the biological sample with 1% to 20% NBF for about 48 hours.

In some embodiments, the method provided herein comprises treating the biological sample with 10% NBF for about 0.1 hour. In some embodiments, the method provided herein comprises treating the biological sample with 10% NBF for about 0.25 hour. In some embodiments, the method provided herein comprises treating the biological sample with 10% NBF for about 0.5 hour. In some embodiments, the method provided herein comprises treating the biological sample with 10% NBF for about 0.75 hour. In some embodiments, the method provided herein comprises treating the biological sample with 10% NBF for about 1 hour. In some embodiments, the method provided herein comprises treating the biological sample with 10% NBF for about 2 hours. In some embodiments, the method provided herein comprises treating the biological sample with 10% NBF for about 3 hours. In some embodiments, the method provided herein comprises treating the biological sample with 10% NBF for about 4 hours. In some embodiments, the method provided herein comprises treating the biological sample with 10% NBF for about 5 hours. In some embodiments, the method provided herein comprises treating the biological sample with 10% NBF for about 6 hours. In some embodiments, the method provided herein comprises treating the biological sample with 10% NBF for about 7 hours. In some embodiments, the method provided herein comprises treating the biological sample with 10% NBF for about 8 hours. In some embodiments, the method provided herein comprises treating the biological sample with 10% NBF for about 9 hours. In some embodiments, the method provided herein comprises treating the biological sample with 10% NBF for about 10 hours. In some embodiments, the method provided herein comprises treating the biological sample with 10% NBF for about 11 hours. In some embodiments, the method provided herein comprises treating the biological sample with 10% NBF for about 12 hours. In some embodiments, the method provided herein comprises treating the biological sample with 10% NBF for about 14 hours. In some embodiments, the method provided herein comprises treating the biological sample with 10% NBF for about 18 hours. In some embodiments, the method provided herein comprises treating the biological sample with 10% NBF for about 20 hours. In some embodiments, the method provided herein comprises treating the biological sample with 10% NBF for about 24 hours. In some embodiments, the method provided herein comprises treating the biological sample with 10% NBF for about 36 hours. In some embodiments, the method provided herein comprises treating the biological sample with 10% NBF for about 48 hours.

In some specific embodiments, the method provided herein comprises treating the biological sample with a crosslinking agent at a temperature of 4° C. for more than 10 hours. In some specific embodiments, the method provided herein comprises treating the biological sample with a crosslinking agent at a temperature of 4° C. for more than 5 hours. In some specific embodiments, the method provided herein comprises treating the biological sample with a crosslinking agent at a temperature of 4° C. for more than 1 hour. In some specific embodiments, the method provided herein comprises treating the biological sample with a crosslinking agent at a temperature of 4° C. for about 5 hours. In some specific embodiments, the method provided herein comprises treating the biological sample with a crosslinking agent at a temperature of 4° C. for about 6 hours. In some specific embodiments, the method provided herein comprises treating the biological sample with a crosslinking agent at a temperature of 4° C. for about 7 hours. In some specific embodiments, the method provided herein comprises treating the biological sample with a crosslinking agent at a temperature of 4° C. for about 8 hours. In some specific embodiments, the method provided herein comprises treating the biological sample with a crosslinking agent at a temperature of 4° C. for about 9 hours. In some specific embodiments, the method provided herein comprises treating the biological sample with a crosslinking agent at a temperature of 4° C. for about 10 hours. In some specific embodiments, the method provided herein comprises treating the biological sample with a crosslinking agent at a temperature of 4° C. for about 11 hours. In some specific embodiments, the method provided herein comprises treating the biological sample with a crosslinking agent at a temperature of 4° C. for about 12 hours.

In some specific embodiments, the method provided herein comprises treating the biological sample with a crosslinking agent at room temperature for less than about 6 hours. In some specific embodiments, the method provided herein comprises treating the biological sample with a crosslinking agent at room temperature for less than about 3 hours. In some specific embodiments, the method provided herein comprises treating the biological sample with a crosslinking agent at room temperature for less than about 1 hour. In some specific embodiments, the method provided herein comprises treating the biological sample with a crosslinking agent at room temperature for less than about 0.5 hour. In some specific embodiments, the method provided herein comprises treating the biological sample with a crosslinking agent at room temperature for about 0.1 hour. In some specific embodiments, the method provided herein comprises treating the biological sample with a crosslinking agent at room temperature for about 0.15 hour. In some specific embodiments, the method provided herein comprises treating the biological sample with a crosslinking agent at room temperature for about 0.2 hour. In some specific embodiments, the method provided herein comprises treating the biological sample with a crosslinking agent at room temperature for about 0.25 hour. In some specific embodiments, the method provided herein comprises treating the biological sample with a crosslinking agent at room temperature for about 0.3 hour. In some specific embodiments, the method provided herein comprises treating the biological sample with a crosslinking agent at room temperature for about 0.35 hour. In some specific embodiments, the method provided herein comprises treating the biological sample with a crosslinking agent at room temperature for about 0.4 hour. In some specific embodiments, the method provided herein comprises treating the biological sample with a crosslinking agent at room temperature for about 0.45 hour. In some specific embodiments, the method provided herein comprises treating the biological sample with a crosslinking agent at room temperature for about 0.5 hour. In some specific embodiments, the method provided herein comprises treating the biological sample with a crosslinking agent at room temperature for about 0.55 hour. In some specific embodiments, the method provided herein comprises treating the biological sample with a crosslinking agent at room temperature for about 0.6 hour. In some specific embodiments, the method provided herein comprises treating the biological sample with a crosslinking agent at room temperature for about 0.65 hour. In some specific embodiments, the method provided herein comprises treating the biological sample with a crosslinking agent at room temperature for about 0.7 hour. In some specific embodiments, the method provided herein comprises treating the biological sample with a crosslinking agent at room temperature for about 0.75 hour. In some specific embodiments, the method provided herein comprises treating the biological sample with a crosslinking agent at room temperature for about 0.8 hour. In some specific embodiments, the method provided herein comprises treating the biological sample with a crosslinking agent at room temperature for about 0.85 hour. In some specific embodiments, the method provided herein comprises treating the biological sample with a crosslinking agent at room temperature for about 0.9 hour. In some specific embodiments, the method provided herein comprises treating the biological sample with a crosslinking agent at room temperature for about 1 hour. In some specific embodiments, the method provided herein comprises treating the biological sample with a crosslinking agent at room temperature for about 1.5 hours. In some specific embodiments, the method provided herein comprises treating the biological sample with a crosslinking agent at room temperature for about 2 hours. In some specific embodiments, the method provided herein comprises treating the biological sample with a crosslinking agent at room temperature for about 2.5 hours. In some specific embodiments, the method provided herein comprises treating the biological sample with a crosslinking agent at room temperature for about 3 hours. In some specific embodiments, the method provided herein comprises treating the biological sample with a crosslinking agent at room temperature for about 3.5 hours. In some specific embodiments, the method provided herein comprises treating the biological sample with a crosslinking agent at room temperature for about 4 hours.

In some specific embodiments, the method provided herein comprises treating the biological sample with a crosslinking agent at a temperature of 40° C. for less than about 6 hours. In some specific embodiments, the method provided herein comprises treating the biological sample with a crosslinking agent at a temperature of 40° C. for less than about 3 hours. In some specific embodiments, the method provided herein comprises treating the biological sample with a crosslinking agent at a temperature of 40° C. for less than about 1 hour. In some specific embodiments, the method provided herein comprises treating the biological sample with a crosslinking agent at a temperature of 40° C. for less than about 0.5 hour. In some specific embodiments, the method provided herein comprises treating the biological sample with a crosslinking agent at a temperature of 40° C. for about 0.1 hour. In some specific embodiments, the method provided herein comprises treating the biological sample with a crosslinking agent at a temperature of 40° C. for about 0.25 hour. In some specific embodiments, the method provided herein comprises treating the biological sample with a crosslinking agent at a temperature of 40° C. for about 0.5 hour. In some specific embodiments, the method provided herein comprises treating the biological sample with a crosslinking agent at a temperature of 40° C. for about 0.75 hour. In some specific embodiments, the method provided herein comprises treating the biological sample with a crosslinking agent at a temperature of 40° C. for about 1 hour.

In some specific embodiments, the method provided herein comprises treating the biological sample with a crosslinking agent at a temperature of 60° C. for less than about 6 hours. In some specific embodiments, the method provided herein comprises treating the biological sample with a crosslinking agent at a temperature of 60° C. for less than about 3 hours. In some specific embodiments, the method provided herein comprises treating the biological sample with a crosslinking agent at a temperature of 60° C. for less than about 1 hour. In some specific embodiments, the method provided herein comprises treating the biological sample with a crosslinking agent at a temperature of 60° C. for less than about 0.5 hour. In some specific embodiments, the method provided herein comprises treating the biological sample with a crosslinking agent at a temperature of 60° C. for about 0.1 hour. In some specific embodiments, the method provided herein comprises treating the biological sample with a crosslinking agent at a temperature of 60° C. for about 0.25 hour. In some specific embodiments, the method provided herein comprises treating the biological sample with a crosslinking agent at a temperature of 60° C. for about 0.5 hour. In some specific embodiments, the method provided herein comprises treating the biological sample with a crosslinking agent at a temperature of 60° C. for about 0.75 hour. In some specific embodiments, the method provided herein comprises treating the biological sample with a crosslinking agent at a temperature of 60° C. for about 1 hour.

In some embodiments, the method provided herein comprises treating the biological sample with a crosslinking agent at a pH of about 6. In some embodiments, the method provided herein comprises treating the biological sample with a crosslinking agent at a pH of about 6.5. In some embodiments, the method provided herein comprises treating the biological sample with a crosslinking agent at a pH of about 7. In some embodiments, the method provided herein comprises treating the biological sample with a crosslinking agent at a pH of about 7.5. In some embodiments, the method provided herein comprises treating the biological sample with a crosslinking agent at a pH of about 8. In some embodiments, the method provided herein comprises treating the biological sample with a crosslinking agent at a pH of about 8.5. In some embodiments, the method provided herein comprises treating the biological sample with a crosslinking agent at a pH of about 9.

In some specific embodiments, the method provided herein comprises treating the biological sample with a crosslinking agent with an effective concentration of less than 1%. In some specific embodiments, the method provided herein comprises treating the biological sample with a crosslinking agent with an effective concentration of more than 50%. In some specific embodiments, the method provided herein comprises treating the biological sample with a crosslinking agent with an effective concentration of 1% to 50%. In some specific embodiments, the method provided herein comprises treating the biological sample with a crosslinking agent with an effective concentration of 2% to 40%. In some specific embodiments, the method provided herein comprises treating the biological sample with a crosslinking agent with an effective concentration of 3% to 30%. In some specific embodiments, the method provided herein comprises treating the biological sample with a crosslinking agent with an effective concentration of 4% to 20%. In some specific embodiments, the method provided herein comprises treating the biological sample with a crosslinking agent with an effective concentration of about 4%. In some specific embodiments, the method provided herein comprises treating the biological sample with a crosslinking agent with an effective concentration of 12%. In some specific embodiments, the method provided herein comprises treating the biological sample with a crosslinking agent with an effective concentration of 24%. In some specific embodiments, the method provided herein comprises treating the biological sample with a crosslinking agent with an effective concentration of 37%.

In some specific embodiments, the method provided herein comprises treating the biological sample with a crosslinking agent with an effective concentration of formaldehyde or equivalence at 1% to 50%. In some specific embodiments, the method provided herein comprises treating the biological sample with a crosslinking agent with an effective concentration of formaldehyde or equivalence at 2% to 40%. In some specific embodiments, the method provided herein comprises treating the biological sample with a crosslinking agent with an effective concentration of formaldehyde or equivalence at 3% to 30%. In some specific embodiments, the method provided herein comprises treating the biological sample with a crosslinking agent with an effective concentration of formaldehyde or equivalence at 4% to 20%. In some specific embodiments, the method provided herein comprises treating the biological sample with a crosslinking agent with an effective concentration of formaldehyde or equivalence at about 4%. In some specific embodiments, the method provided herein comprises treating the biological sample with a crosslinking agent with an effective concentration of formaldehyde or equivalence at 12%. In some specific embodiments, the method provided herein comprises treating the biological sample with a crosslinking agent with an effective concentration of formaldehyde or equivalence at 24%. In some specific embodiments, the method provided herein comprises treating the biological sample with a crosslinking agent with an effective concentration of formaldehyde or equivalence at 37%.

In certain embodiments, the method provided herein comprises treating the biological sample with a crosslinking agent in an isotonic buffer with a pH at about 6 and an effective concentration of formaldehyde or equivalent less than 12%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent less than 12%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent less than 1 2%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent less than 12%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent less than 1 2%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent less than 12%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent less than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent less than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent less than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent less than 12%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent less than 12%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent less than 1 2%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent more than 12%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent more than 1 2%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent more than 12%, and wherein the temperature is 4° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent more than 12%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent more than 12%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent more than 12%, and wherein the temperature is room temperature. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent more than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent more than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent more than 12%, and wherein the temperature is 40° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent more than 12%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 7 and an effective concentration of formaldehyde or equivalent more than 12%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 8 and an effective concentration of formaldehyde or equivalent more than 12%, and wherein the temperature is 60° C. In one embodiment, the crosslinking lasts for less than about 0.5 hour. In one embodiment, the crosslinking lasts for less than about 1 hour. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

In certain embodiments, the method provided herein comprises treating the biological sample with a crosslinking agent in an isotonic buffer with a pH at about 6 and an effective concentration of formaldehyde or equivalent more than 12%, and wherein the temperature is 4° C. In one embodiment, the crosslinking lasts for more than about 1 hour. In one embodiment, the crosslinking lasts for more than about 6 hours. In one embodiment, the crosslinking lasts for more than about 12 hours. In one embodiment, the crosslinking lasts for more than about 18 hours. In one embodiment, the crosslinking lasts for more than about 24 hours. In one embodiment, the crosslinking lasts for more than about 30 hours. In one embodiment, the crosslinking lasts for more than about 36 hours. In one embodiment, the crosslinking lasts for more than about 48 hours.

The method provided herein comprises detecting target nucleic acids. In some embodiments, the target nucleic acids are DNAs. In other embodiments, the target nucleic acids are RNAs.

In some embodiments, the method provided herein is for preparing a biological sample for simultaneously detecting target RNAs and target proteins, comprising (i) incubating the biological sample with a primary antibody; (ii) treating the biological sample with a crosslinking agent after (i), and (iii) detecting the target RNAs by a hybridization-based method after (ii). In one embodiment, the method comprises detecting the target RNAs by ISH In one embodiment, the method comprises detecting the target RNAs by molecular beacons (see Tyagi et al., Nat Biotechnol. 4(3):303-308 (1996)). In one embodiment, the method comprises detecting the target RNAs by forced intercalation (FIT) probes (see Köhler et al., Chembiochem. 6(1):69---77(2005)). In some embodiments, the method provided herein is for preparing a biological sample for simultaneously detecting target RNAs and target proteins, comprising (i) incubating the biological sample with a primary antibody; (ii) treating the biological sample with a crosslinking agent after (i), and (iii) detecting the target RNAs by an aptamer-based method after (ii). In one embodiment, the method comprises detecting the target RNAs by an aptamer “Spinach” (see Paige et al., Science, 333(6042):642-646 (2011)). In some embodiments, the method provided herein is for preparing a biological sample for simultaneously detecting target RNAs and target proteins, comprising (i) incubating the biological sample with a primary antibody; (ii) treating the biological sample with a crosslinking agent after (i); and (iii) detecting the target RNAs by particle-associated-hybridization-based probes after (ii). In one embodiment, the method comprises detecting the target RNAs by a gold nanoparticle-quantum dot-probe. In some embodiments, the method provided herein is for preparing a biological sample for simultaneously detecting a target RNAs and target proteins, comprising (i) incubating the biological sample with a primary antibody; (ii) treating the biological sample with a crosslinking agent after (i); and (iii) detecting the target RNAs by incorporating visualizable moieties directly into the target RNAs (see Jao et al., Proc Natl Acad Sci. 105(41):15779-15784(2008)). In some embodiments, the method provided herein is for preparing a biological sample for simultaneously detecting target RNAs and target proteins, comprising (i) incubating the biological sample with a primary antibody; (ii) treating the biological sample with a crosslinking agent after (i); and (iii) detecting the target RNAs by an RNA-binding protein. In one embodiment, the method comprises detecting the target RNAs by fluorescent protein-fused version of the bacteriophage MS2 coat protein (MCP) (see Park et al., Science. 343(6169):422-424(2014)). In one embodiment, the method comprises detecting the target RNAs by a Pumilio-based method (see Kellermann et al., Chembiochem. 14(2):200-204(2013)).

In some embodiments, the method provided herein is for preparing a biological sample for simultaneously detecting target DNAs and target proteins, comprising (i) incubating the biological sample with a primary antibody; (ii) treating the biological sample with a crosslinking agent after (i); and (iii) detecting the target DNAs by a hybridization-based method after (ii). In some specific embodiments, the method comprises detecting the target DNAs by chromogenic ISH. In other specific embodiments, the method comprises detecting the target DNAs by fluorescence ISH.

In some embodiments, the method for preparing a biological sample for simultaneously detecting a target nucleic acid and a target protein comprises the biological sample of various sources. In one embodiment, the biological sample is a tissue specimen or is derived from a tissue specimen. In one embodiment, the biological sample is a blood sample or is derived from a blood sample. In one embodiment, the biological sample is a cytological sample or is derived from a cytological sample. In one embodiment, the biological sample is cultured cells. In another embodiment, the biological sample is a sample containing exosomes.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a nucleic acid target that is a biomarker indicative of a pathology.

It is understood by those skilled in the art that any of a number of suitable sample types can be used for detecting a target nucleic acid and a target protein using methods provided herein. The sample for use in the methods provided herein is generally a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a cell, a primary cell culture, a cell line, a tissue, an organ, an organoid, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, e.g., Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, e.g., Cecil Textbook of Medicine, Bennett and Plum, eds., 20th ed., WB Saunders, Philadelphia (1996); Colposcopy and Treatment of Cervical IntraepithelialNeoplasia: A Beginner’s Manual, Sellors and Sankaranarayanan, eds., International Agency for Research on Cancer, Lyon, France (2003); Kalaf and Cooper, J. Clin. Pathol. 60:449-455 (2007); Brown and Trimble, BestPract. Res. Clin. Obslet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In particular embodiments, the sample is a tissue specimen or is derived from a tissue specimen. In some embodiments, the tissue specimen is FFPE. In some embodiments, the tissue specimen is fresh frozen. In some embodiments, the tissue specimen is prepared with a fixative. In some embodiments, the tissue specimen is prepared with a crosslinking fixative. In other particular embodiments, the sample is a blood sample or is derived from a blood sample. In still other particular embodiments, the sample is a cytological sample or is derived from a cytological sample.

### 6.3 Methods of Simultaneously Detecting a Target Nucleic Acid And a Target Protein

In another aspect, provided herein is a method for simultaneously detecting a target nucleic acid and a target protein in a biological sample, comprising: (i) incubating the biological sample with a primary antibody; (ii) treating the biological sample with a crosslinking agent; (iii) treating the biological sample with a protease; (iv) detecting the target nucleic acid by in situ hybridization; and (v) detecting the target protein by incubating the biological sample with a secondary antibody or other labeling methods.

Beyond the description in Section 6.2 above, the ISH comprises hybridizing the target nucleic acid with one or more target probe(s). Methods for in situ detection of nucleic acids are well known to those skilled in the art (see, e.g., US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, in a sample, in particular a portion or section of tissue or cells (in situ). The probe types can be double stranded DNA (dsDNA), single stranded DNA (ssDNA), single stranded complementary RNA (sscRNA), and/or synthetic oligonucleotides.

In some embodiments, the ISH provided herein comprises providing at least one set of one or more target probe(s) capable of hybridizing to said target nucleic acid, providing a signal-generating complex capable of hybridizing to said set of one or more target probe(s), said signal-generating complex comprises a nucleic acid component capable of hybridizing to said set of one or more target probe(s) and a label probe; hybridizing said target nucleic acid to said set of one or more target probe(s); and capturing the signal-generating complex to said set of one or more target probe(s) and thereby capturing the signal-generating complex to said target nucleic acid.

In some embodiments, each set of one or more target probe(s) comprises a single probe. In other embodiments, each set of one or more target probe(s) comprises two probes. In yet other embodiments, each set of one or more target probe(s) comprises more than two probes.

In some embodiments, when each set of target probes comprises a single target probe, a signal-generating complex is formed when the single target probe is bound to the target nucleic acid. In other embodiments, when each set of target probes comprise two target probes, a signal-generating complex is formed when both members of a target probe pair are bound to the target nucleic acid.

In some specific embodiments, the ISH used herein is RNAscope®, which is described in more detail in, e.g., U.S. Pat. Nos. 7,709,198, 8,604,182, and 8,951,726. Specifically, RNAscope® describes using specially designed oligonucleotide probes in combination with a branched-DNA-like signal-generating complex to reliably detect RNA as small as 1 kilobase at single-molecule sensitivity under standard bright-field microscopy (Anderson et al., J. Cell. Biochem. 117(10):2201-2208 (2016); Wang el al., J. Mol. Diagn. 14(1):22-29 (2012)).

In some embodiments, each target probe comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, one set of one or more target probe(s) is used to detect a target nucleic acid. In other embodiments, two or more sets of one or more target probe(s) are used to detect a target nucleic acid. In some embodiments, two sets of one or more target probe(s) are used to detect a target nucleic acid. In some embodiments, three sets of one or more target probe(s) are used to detect a target nucleic acid. In some embodiments, four sets of one or more target probe(s) are used to detect a target nucleic acid. In some embodiments, five sets of one or more target probe(s) are used to detect a target nucleic acid. In some embodiments, six sets of one or more target probe(s) are used to detect a target nucleic acid. In some embodiments, seven sets of one or more target probe(s) are used to detect a target nucleic acid. In some embodiments, eight sets of one or more target probe(s) are used to detect a target nucleic acid. In some embodiments, nine sets of one or more target probe(s) are used to detect a target nucleic acid. In some embodiments, ten sets of one or more target probe(s) are used to detect a target nucleic acid. In some embodiments, more than 10 sets of one or more target probe(s) are used to detect a target nucleic acid. In some embodiments, more than 15 sets of one or more target probe(s) are used to detect a target nucleic acid. In some embodiments, more than 20 sets of one or more target probe(s) are used to detect a target nucleic acid. In some embodiments, more than 30 sets of one or more target probe(s) are used to detect a target nucleic acid.

In some embodiments, the method provided herein is for detecting multiple nucleic acid targets. In some embodiments, all of the multiple nucleic acid targets comprise less than 100 nucleotides. In other embodiments, some of the nucleic acid targets comprise less than 100 nucleotides, while other targets comprise more than 100 nucleotides. In other embodiments, some of the multiple nucleic acid targets comprise more than 1000 nucleotides.

As used herein, a “target probe” is a polynucleotide that is capable of hybridizing to a target nucleic acid and capturing or binding a label probe or signal-generating complex (SGC) component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. The target probe is generally single stranded so that the complementary sequence is available to hybridize with a corresponding target nucleic acid, label probe, amplifier, pre-amplifier or pre-pre-amplifier. In some embodiments, the target probes are provided as a pair.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, pre-amplifier, or pre-pre-amplifier in an SGC.

In some embodiments, the SGC provided herein comprises additional comments such an amplifier, a pre-amplifier, and/or a pre-pre-amplifier.

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. Publications 2008/0038725 and 2009/0081688, each of which is incorporated by reference.

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference.

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated by reference.

A label is typically used in ISH for detecting target nucleic acid. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752, 3,939,350; 3,996,345; 4,277,437; 4,275,149; and 4,366,241. Many labels are commercially available and can be used in methods and assays of the disclosure, including detectable enzyme/substrate combinations (Pierce, Rockford IL; Santa Cruz. Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al.,Microbiol. Rev. 55:335-348 (1991)), and such enzymes with known chromogenic or fluorogenic substrates can readily be adapted for use in methods provided herein.

Various chromogenic or fluorogenic substrates to produce detectable signal are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-l-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-l-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis (2-amino- 2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase, 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β- D-glucopyranoside for β-glucosidase; and 4-methylumbelliferyl-α- D-galactopyranoside for α-galactosidase. Exemplary enzymes and substrates for producing a detectable signal are also described, for example, in U.S. Publication 2012/0100540. Various detectable enzyme substrates, including chromogenic or fluorogenic substrates, are well known and commercially available (Pierce, Rockford IL; Santa Cruz Biotechnology, Dallas TX, Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb, ¹⁷²Yb, ¹⁷³Yb, ¹⁷⁴Yb, ¹⁷⁵Lu, and ¹⁷⁶Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other, and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. Publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-l-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can be used for concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the disclosure are well known in the art (see, e.g., Johnson and Spence, “Molecular Probes Handbook, a Guide to FluorescentProbes and Labeling Technologies, 11th ed., Life Technologies (2010)).

Well known methods such as microscopy, cytometry (e.g., mass cytometry, cytometry by time of flight (CyTOF), flow cytometry), or spectroscopy can be utilized to visualize chromogenic, fluorescent, or metal detectable signal associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of target nucleic acids. Generally, the labels are conjugated to the label probe by a chemical linker that is cleavable. Methods of conjugating a label to a label probe so that the label is cleavable are well known to those skilled in the art (see, e.g., Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlingame CA). Various cleavable moieties can be included in the linker so that the label can be cleaved from the label probe. Such cleavable moieties include groups that can be chemically, photochemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xa (cleavage site IEGR/), tobacco etch virus protease (cleavage site ENLYFQ/G), and thrombin (cleavage site LVPR/GS) (see, e.g., Oxford Genetics, Oxford, UK). Another cleavable moiety can be, for example, uracil-DNA (DNA containing uracil), which can be cleaved by uracil-DNA glycosylase (UNG) (see, e.g., Sidorenko et al., FEBS Lett. 582(3):410—404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

In some embodiments, the method provided herein comprises using a labeled primary antibody, thus eliminating the needs for performing other IHC steps. In specific embodiments, the primary antibody is labeled with chromogenic labels. In specific embodiments, the primary antibody is labeled with florescent labels. In specific embodiments, the primary antibody is labeled with polynucleotide(s). In specific embodiments, the primary antibody is labeled by NHS (succinimidyl) ester method. In specific embodiments, the primary antibody is labeled by isothiocyanate method. In specific embodiments, the primary antibody is labeled by carbodiimide method. In specific embodiments, the primary antibody is labeled by two-tag method (a catalyst and its substrate). In specific embodiments, the primary antibody is labeled by periodate method.

The post-primary-antibody crosslinking can be adapted for use with fluorescent-based detection, combined with the BaseScope™ signal amplification system (see Baker et al., Nature Communication 8:1998 (2017)), or combined with other nucleic acid detection methods using similar protocols. In some embodiments, the method provided herein is for preparing a biological sample for simultaneously detecting a target RNA and a target protein, comprising (i) incubating the biological sample with a primary antibody; (ii) treating the biological sample with a crosslinking agent after (i); and (iii) detecting the target RNA by a hybridization-based method after (ii). In one embodiment, the method comprises detecting the target RNA by ISH. In one embodiment, the method comprises detecting the target RNA by molecular beacons (see Tyagi et al., Nat Biotechnol. 4(3):303-308 (1996)). In one embodiment, the method comprises detecting the target RNA by forced intercalation (FIT) probes (see Köhler et al., Chembiochem. 6(1):69---77(2005)). In some embodiments, the method provided herein is for preparing a biological sample for simultaneously detecting a target RNA and a target protein, comprising (i) incubating the biological sample with a primary antibody; (ii) treating the biological sample with a crosslinking agent after (i); and (iii) detecting the target RNA by an aptamer-based method after (ii). In one embodiment, the method comprises detecting the target RNA by an aptamer “Spinach” (see Paige et al., Science, 333(6042):642-646 (2011)). In some embodiments, the method provided herein is for preparing a biological sample for simultaneously detecting a target RNA and a target protein, comprising (i) incubating the biological sample with a primary antibody; (ii) treating the biological sample with a crosslinking agent after (i); and (iii) detecting the target RNA by particle-associated-hybridization-based probes after (ii). In one embodiment, the method comprises detecting the target RNA by a gold nanoparticle-quantum dot-probe. In some embodiments, the method provided herein is for preparing a biological sample for simultaneously detecting a target RNA and a target protein, comprising (i) incubating the biological sample with a primary antibody; (ii) treating the biological sample with a crosslinking agent after (i); and (iii) detecting the target RNA by incorporating visualizable moieties directly into the target RNA (see Jao et al., Proc Natl Acad Sci. 105(41):15779-15784(2008)). In some embodiments, the method provided herein is for preparing a biological sample for simultaneously detecting a target RNA and a target protein, comprising (i) incubating the biological sample with a primary antibody; (ii) treating the biological sample with a crosslinking agent after (i); and (iii) detecting the target RNA by an RNA-binding protein. In one embodiment, the method comprises detecting the target RNA by fluorescent protein-fused version of the bacteriophage MS2 coat protein (MCP) (see Park et al., Science. 343(6169):422-424(2014)). In one embodiment, the method comprises detecting the target RNA by a Pumilio-based method (see Kellermann et al., Chembiochem. 14(2):200-204(2013))

Since DNA detections may employ similar protease and hybridization conditions that can disrupt antigen-antibody binding, the cross-linking approach described here also enables protein-DNA co-detection. In some embodiments, the method provided herein is for preparing a biological sample for simultaneously detecting a target DNA and a target protein, comprising (i) incubating the biological sample with a primary antibody; (ii) treating the biological sample with a crosslinking agent after (i); and (iii) detecting the target DNA by a hybridization-based method after (ii). In one embodiment, the method comprises detecting the target DNA by chromogenic ISH. In one embodiment, the method comprises detecting the target DNA by fluorescence ISH.

The method provided herein is a valuable research tool as well as diagnostic tool. In some embodiments, the method provided herein is used for mapping spatial organization in a complex tissue. In some specific embodiments, the method provided herein is used for identifying cell types and new cell types. In some specific embodiments, the method provided herein is used for identifying cellular states. In other specific embodiments, the method provided herein is used for identifying cell types and new cell types in a tumor microenvironment. In some specific embodiments, the method provided herein is used for identifying cellular states in a tumor microenvironment.

In some embodiments, the method provided herein is used for detecting altered gene expression in diseased cells and tissues. In some specific embodiments, the method provided herein is used for localizing altered gene expression in specific cell types and understanding tumor heterogeneity. In some specific embodiments, the method provided herein is used for studying tumor-immune cell interactions. In some embodiments, the method provided herein is used for detecting biomarkers for cancer diagnosis and prognosis. In some embodiments, the method provided herein is used for detecting therapeutic targets for cancer treatment. In some embodiments, the method provided herein is used for facilitating the validation of novel antibodies.

### 6.4 A Kit for Simultaneously Detecting Target Nucleic Acids and Target Proteins

In yet another aspect, provided herein is a kit for performing the various methods described in Sections 6.2 and 6.3 above.

In another aspect, provided herein is a kit for simultaneously detecting a target nucleic acid and a target protein in a biological sample, comprising: (i) a crosslinking agent; and (ii) an instruction indicating that the crosslinking agent is used after incubating the biological sample with a primary antibody that detects the target protein. In some embodiments, the kit further comprises a protease. In some embodiments, the kit further comprises an agent for detecting the target nucleic acid and/or an agent for detecting the target protein.

In yet another aspect, provided herein is a kit for simultaneously detecting a target nucleic acid and a target protein in a biological sample, comprising: (i) a crosslinking agent; and (ii) a protease. In some embodiments, the kit further comprises an instruction indicating that the crosslinking agent is used before the protease and the crosslinking agent is used after a primary antibody that detects the target protein. In some embodiments, the kit further comprises an agent for detecting the target nucleic acid and/or an agent for detecting the target protein.

In certain embodiments, the crosslinking agent in the kit is a fixative. In specific embodiments, the crosslinking agent in the kit is formaldehyde. In specific embodiments, the crosslinking agent in the kit is glutaraldehyde. In specific embodiments, the crosslinking agent in the kit is acrolein. In specific embodiments, the crosslinking agent in the kit is osmium tetroxide. In specific embodiments, the crosslinking agent in the kit is a type of permanganate fixative. In one embodiment, the crosslinking agent in the kit is potassium permanganate. In specific embodiments, the crosslinking agent in the kit is a type of dichromate fixative. In one embodiment, the crosslinking agent in the kit is potassium dichromate. In specific embodiments, the crosslinking agent in the kit is chromic acid.

In some embodiments, the kit provided herein comprises a crosslinking agent is a mixture of crosslinking fixatives. In some embodiments, the crosslinking agent is a mixture solution of two or more fixatives, selected from a list of formaldehyde, glutaraldehyde, acrolein, osmium tetroxide, permanganate fixative, dichromate fixative, and chromic acid. In one embodiment, the crosslinking agent is Bouin’s fixative, which is a solution of picric acid, formaldehyde, and acetic acid. In one embodiment, the crosslinking agent is a mixture of formaldehyde and glutaraldehyde. In one embodiment, the crosslinking agent is FAA, which is a solution of ethanol, acetic acid, and formaldehyde. In one embodiment, the crosslinking agent is periodate-lysine-paraformaldehyde (PLP), which is a solution of paraformaldehyde, L-lysine, and INaO4. In one embodiment, the crosslinking agent is phosphate buffered formalin (PBF). In one embodiment, the crosslinking agent is formal calcium, which is a solution of formaldehyde and calcium chloride. In one embodiment, the crosslinking agent is formal saline, which is a solution of formaldehyde and sodium chloride. In one embodiment, the crosslinking agent is zinc formalin, which is a solution of formaldehyde and zinc sulphate. In one embodiment, the crosslinking agent is Helly’s fixative, which is a solution of formaldehyde, potassium dichromate, sodium sulphate, and mercuric chloride. In one embodiment, the crosslinking agent is Hollande’s fixative, which is a solution of formaldehyde, copper acetate, picric acid, and acetic acid. In one embodiment, the crosslinking agent is Gendre’s solution, which is a solution of formaldehyde, ethanol, picric acid, and acetic acid glacial. In one embodiment, the crosslinking agent is alcoholic formalin, which is a solution of formaldehyde, ethanol, and calcium acetate. In one embodiment, the crosslinking agent is formol acetic alcohol, which is a solution of formaldehyde, acetic acid glacial, and ethanol. In one embodiment, the crosslinking agent is a mixture of fixatives, wherein at least one fixative of the mixture is formaldehyde or glutaraldehyde. In one embodiment, the crosslinking agent is fixatives that are not used at the same time but separately or consecutively, wherein at least one fixative is formaldehyde or glutaraldehyde.

In some embodiments, the kit provided herein comprises a crosslinking agent with two or more agents, which are not applied at the same time but separately or consecutively and are selected from a list of formaldehyde, glutaraldehyde, acrolein, osmium tetroxide, permanganate fixative, dichromate fixative, and chromic acid.

In some embodiments, the kit provided herein comprises an instruction indicating treating the biological sample with a crosslinking agent at a temperature of about 0° C. to about 100° C. In some embodiments, the kit provided herein comprises an instruction indicating treating the biological sample with a crosslinking agent at a temperature of about 1° C. to about 90° C. In some embodiments, the kit provided herein comprises an instruction indicating treating the biological sample with a crosslinking agent at a temperature of about 2° C. to about 80° C. In some embodiments, the kit provided herein comprises an instruction indicating treating the biological sample with a crosslinking agent at a temperature of about 3° C. to about 70° C. In some embodiments, the kit provided herein comprises an instruction indicating treating the biological sample with a crosslinking agent at a temperature of about 4° C. to about 60° C.

In some embodiments, the kit provided herein comprises an instruction indicating treating the biological sample with a crosslinking agent at a temperature of about 1° C. In some embodiments, the kit provided herein comprises an instruction indicating treating the biological sample with a crosslinking agent at a temperature of about 2° C. In some embodiments, the kit provided herein comprises an instruction indicating treating the biological sample with a crosslinking agent at a temperature of about 3° C. In some embodiments, the kit provided herein comprises an instruction indicating treating the biological sample with a crosslinking agent at a temperature of about 4° C. In some embodiments, the kit provided herein comprises an instruction indicating treating the biological sample with a crosslinking agent at a temperature of about 5° C. In some embodiments, the kit provided herein comprises an instruction indicating treating the biological sample with a crosslinking agent at a temperature of about 6° C. In some embodiments, the kit provided herein comprises an instruction indicating treating the biological sample with a crosslinking agent at a temperature of about 7° C. In some embodiments, the kit provided herein comprises an instruction indicating treating the biological sample with a crosslinking agent at a temperature of about 8° C. In some embodiments, the kit provided herein comprises an instruction indicating treating the biological sample with a crosslinking agent at a temperature of about 9° C. In some embodiments, the kit provided herein comprises an instruction indicating treating the biological sample with a crosslinking agent at a temperature of about 10° C. In some embodiments, the kit provided herein comprises an instruction indicating treating the biological sample with a crosslinking agent at a temperature of about 11° C. In some embodiments, the kit provided herein comprises an instruction indicating treating the biological sample with a crosslinking agent at a temperature of about 12° C. In some embodiments, the kit provided herein comprises an instruction indicating treating the biological sample with a crosslinking agent at a temperature of about 13° C. In some embodiments, the kit provided herein comprises an instruction indicating treating the biological sample with a crosslinking agent at a temperature of about 14° C. In some embodiments, the kit provided herein comprises an instruction indicating treating the biological sample with a crosslinking agent at a temperature of about 15° C. In some embodiments, the kit provided herein comprises an instruction indicating treating the biological sample with a crosslinking agent at a temperature of about 16° C. In some embodiments, the kit provided herein comprises an instruction indicating treating the biological sample with a crosslinking agent at a temperature of about 17° C. In some embodiments, the kit provided herein comprises an instruction indicating treating the biological sample with a crosslinking agent at a temperature of about 18° C. In some embodiments, the kit provided herein comprises an instruction indicating treating the biological sample with a crosslinking agent at a temperature of about 19° C. In some embodiments, the kit provided herein comprises an instruction indicating treating the biological sample with a crosslinking agent at a temperature of about 20° C. In some embodiments, the kit provided herein comprises an instruction indicating treating the biological sample with a crosslinking agent at a temperature of about 21° C. In some embodiments, the kit provided herein comprises an instruction indicating treating the biological sample with a crosslinking agent at a temperature of about 22° C. In some embodiments, the kit provided herein comprises an instruction indicating treating the biological sample with a crosslinking agent at a temperature of about 23° C. In some embodiments, the kit provided herein comprises an instruction indicating treating the biological sample with a crosslinking agent at a temperature of about 24° C. In some embodiments, the kit provided herein comprises an instruction indicating treating the biological sample with a crosslinking agent at a temperature of about 25° C. In some embodiments, the kit provided herein comprises an instruction indicating treating the biological sample with a crosslinking agent at a temperature of about 26° C. In some embodiments, the kit provided herein comprises an instruction indicating treating the biological sample with a crosslinking agent at a temperature of about 27° C. In some embodiments, the kit provided herein comprises an instruction indicating treating the biological sample with a crosslinking agent at a temperature of about 28° C. In some embodiments, the kit provided herein comprises an instruction indicating treating the biological sample with a crosslinking agent at a temperature of about 29° C. In some embodiments, the kit provided herein comprises an instruction indicating treating the biological sample with a crosslinking agent at a temperature of about 30° C. In some embodiments, the kit provided herein comprises an instruction indicating treating the biological sample with a crosslinking agent at a temperature of about 35° C. In some embodiments, the kit provided herein comprises an instruction indicating treating the biological sample with a crosslinking agent at a temperature of about 40° C. In some embodiments, the kit provided herein comprises an instruction indicating treating the biological sample with a crosslinking agent at a temperature of about 45° C. In some embodiments, the kit provided herein comprises an instruction indicating treating the biological sample with a crosslinking agent at a temperature of about 50° C. In some embodiments, the kit provided herein comprises an instruction indicating treating the biological sample with a crosslinking agent at a temperature of about 55° C. In some embodiments, the kit provided herein comprises an instruction indicating treating the biological sample with a crosslinking agent at a temperature of about 60° C. In some embodiments, the kit provided herein comprises an instruction indicating treating the biological sample with a crosslinking agent at a temperature of about 65° C. In some embodiments, the kit provided herein comprises an instruction indicating treating the biological sample with a crosslinking agent at a temperature of about 70° C. In some embodiments, the kit provided herein comprises an instruction indicating treating the biological sample with a crosslinking agent at a temperature of about 75° C. In some embodiments, the kit provided herein comprises an instruction indicating treating the biological sample with a crosslinking agent at a temperature of about 80° C. In some embodiments, the kit provided herein comprises an instruction indicating treating the biological sample with a crosslinking agent at a temperature of about 85° C. In some embodiments, the kit provided herein comprises an instruction indicating treating the biological sample with a crosslinking agent at a temperature of about 90° C. In some embodiments, the kit provided herein comprises an instruction indicating treating the biological sample with a crosslinking agent at a temperature of about 95° C. In some embodiments, the kit provided herein comprises an instruction indicating treating the biological sample with a crosslinking agent at a temperature of about 100° C.

In some embodiments, the kit further comprises a tool for obtaining a biological sample from a subject. In certain embodiments, the biological sample is a tissue specimen or is derived from a tissue specimen. In certain embodiments, the biological sample is a blood sample or is derived from a blood sample. In certain embodiments, the biological sample is a cytological sample or is derived from a cytological sample.

In a specific embodiment, the kit provided herein comprises agents for performing RNAscope® as described in more detail in, e.g., U.S. Pat. Nos. 7,709,198, 8,604,182, and 8,951,726. In some embodiments, the kit comprises at least one set of one or more target probe(s) capable of hybridizing to a target nucleic acid; a signal-generating complex capable of hybridizing to said set of one or more target probe(s), wherein said signal-generating complex comprises a label probe and a nucleic acid component capable of hybridizing to said set of one or more target probe(s).

In some embodiments, the target probe(s) comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, the kit further comprises signal-generating complex as described in Section 6.3 above, which may incudes label probe, amplifier, pre-amplifier, and/or pre-pre-amplifier.

In some embodiments, the kit further comprises other agents or materials for performing ISH, including fixing agents and agents for treating samples for preparing hybridization, agents for washing the samples, and so on.

In some embodiments, the kit further comprises other agents or materials for performing IHC, including blocking buffers, secondary antibody, IHC labels, agents for washing the samples, and so on.

The kit may further comprise “packaging material” which refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts. Labels or inserts include information on a condition, disorder, disease, or symptom for which the kit component may be used for. Labels or inserts can include instructions for a clinician or for a subject to use one or more of the kit components in a method, treatment protocol, or therapeutic regimen.

In some embodiments, the kit provided herein is used for mapping spatial organization in a complex tissue. In some specific embodiments, the kit provided herein is used for identifying cell types and new cell types. In some specific embodiments, the kit provided herein is used for identifying cellular states. In other specific embodiments, the kit provided herein is used for identifying cell types and new cell types in a tumor microenvironment. In some specific embodiments, the kit provided herein is used for identifying cellular states in a tumor microenvironment.

In some embodiments, the kit provided herein is used for detecting altered gene expression in diseased cells and tissues. In some specific embodiments, the kit provided herein is used for localizing altered gene expression in specific cell types and understanding tumor heterogeneity. In some specific embodiments, the kit provided herein is used for studying tumor-immune cell interactions. In some embodiments, the kit provided herein is used for detecting biomarkers for cancer diagnosis and prognosis. In some embodiments, the kit provided herein is used for detecting therapeutic targets for cancer treatment. In some embodiments, the kit provided herein is used for facilitating the validation of novel antibodies.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1 ISH/IHC Integrated Co-Detection Workflow

FIG. 1A and FIG. 1B illustrate steps for a sequential ISH-IHC workflow and an integrated dual ISH-IHC work-flow. The original sequential dual ISH-IHC workflow begins with standard RNAscope^{®} pretreatment and completes ISH staining before beginning IHC staining (see FIG. 1A). In contrast, the integrated ISH-IHC co-detection workflow begins with pretreatment only up to the target retrieval. Then, IHC primary antibody is bound to its antigens in tissues/cells and crosslinked, followed by protease treatment and RNAscope^{®} staining. Once RNAscope^{®} staining is completed, IHC detection re-commences with exposure to secondary antibody and staining (see FIG. 1B). In theory, using directly labeled primary antibody for IHC could shorten this integrated workflow by removing the need for an exposure to a secondary antibody.

### 7.2 Post-Primary Crosslinking Preserved or Improved IHC Signal After Exposure to Hybridization Buffer or Protease Treatment

Crosslinking protected CD20 IHC signal against the stress of the formamide-based reagent in the hybridization buffer. FIGS. 2A-2C show sections of FFPE human tonsil tissue that were subject to IHC staining with the Leica Bond Polymer Refine Detection kit. As a baseline, IHC for CD20 detection without any interference from ISH displayed clear signal (see FIG. 2A). A section, after an incubation with CD20 antibody, was exposed to hybridization buffer for 30 minutes at room temperature, followed by the remaining IHC staining. Exposure to the hybridization buffer noticeably diminished the CD20 IHC signal (see FIG. 2B). Post-primary crosslinking was achieved by incubating tissue with commercially available 10% NBF for 30 minutes at room temperature and it improved IHC resistance to the effect of hybridization buffer (see FIG. 2C).

Similarly, crosslinking also improved CD20 IHC signal resistance to protease treatment. Sections of human tonsil were IHC stained to detect CD20 protein using the Leica Bond Polymer Refine Detection kit, and the staining yielded clear signal (see FIG. 3A). Following incubation with the primary antibody, slides were exposed to protease treatment for 15 minutes at 40° C., resulting in marked decrease of IHC signal (see FIG. 3B). Crosslinking tissue for 30 minutes at room temperature with 10% NBF after the incubation of the primary antibody and prior to protease treatment ameliorated the effects of the protease on IHC signal (see FIG. 3C).

For some other antibodies of interest, post-primary crosslinking not only protected IHC signal but also enhanced IHC signal. One example was a CD8 antibody. Sections of human tonsil were IHC stained to detect CD8 using the Leica Bond Polymer Refine Detection kit (see FIG. 4A and FIG. 4D). Following incubation with the CD8 antibody, tissues were exposed to either hybridization buffer for 30 minutes at room temperature (see FIG. 4B), or protease treatment for 15 minutes at 40° C. (see FIG. 4E), resulting in a noticeable decrease of IHC signal. Crosslinking tissue for 30 minutes at room temperature with 10% NBF after the incubation of the primary antibody and prior to either the incubation of the hybridization buffer (see FIG. 4C) or protease treatment (see FIG. 4F) improved CD8 IHC signal when compared to the standard IHC protocol (see FIG. 4A and FIG. 4D, respectively).

### 7.3 ISH/IHC Integrated Co-detection Workflow Successfully Yielded Strong ISH and IHC Signal

As a baseline for IHC or ISH alone, sections of FFPE human head and neck cancer were subject to IHC staining against CD8 with the Leica Bond Polymer Refine Detection kit, detection of IHC signal were visible (see FIG. 5A). Separately, human peptidylprolyl isomerase B (Hs-PPIB) ISH staining using the RNAscope® 2.5 LS Red kit alone was carried out, and ISH signal were also detected (see FIG. 5B). However, using the sequential ISH-IHC workflow (see FIG. 1A), where sections were subject to Hs-PPIB RNAscope® 2.5 LS Red ISH detection followed immediately by CD8 IHC staining with the Leica Bond Polymer Refine detection kit, IHC signal was significantly decreased (see FIG. 5C). In contrast, using the integrated co-detection workflow (see FIG. 1B), tissue was crosslinked after the exposure to primary antibody as described above. Tissue was then stained for Hs-PPIB with the RNAscope® 2.5 LS Red assay, and subsequently underwent the remaining IHC staining steps. Therefore, the integrated ISH/IHC workflow helped preserve antigen-antibody binding and, in the case of CD8, improved IHC signal (see FIG. 5D).

More than one difference exists between the sequential and ISH/IHC integrated co-detection workflow. To further specify which difference(s) are responsible for better ISH and IHC signals with the ISH/IHC integrated co-detection workflow, another set of experiments were carried out. Sections of FFPE human tonsil tissue were subject to IHC staining against CD20 with the Leica Bond Polymer Refine Detection kit followed by Green chromogen, resulting in CD20 protein detection (see FIG. 6A) or ISH staining for Hs-PPIB with the RNAscope® 2.5 LS Red kit, resulting in RNA detection (see FIG. 6B). Alternatively, sections were subjected to RNAscope® pretreatment including a 15-minute incubation with protease at 40° C. prior to IHC staining, which resulted in decreased IHC signal (see FIG. 6C). Using the ISH/IHC integrated co-detection workflow (see FIG. 1B), following exposure to primary antibody, tissue was crosslinked as described above. ISH staining against Hs-PPIB with the RNAscope® 2.5 LS Red assay was carried out, and the remaining IHC staining steps (secondary antibody, chromogenic detection, and counter staining) were subsequently carried out. Crosslinking within the integrated ISH/IHC workflow helped to preserve antigen-antibody binding and improved IHC signal (see FIG. 6E). In contrast, performing the ISH/IHC integrated co-detection workflow without the additional crosslinking step resulted in uniform loss of CD20 IHC signal, thus highlighting the importance of the post-primary crosslinking (see FIG. 6D).

### 7.4 Parameters for Temperatures and Treatment Durations for the Crosslinking in the ISH/IHC Integrated Co-Detection Workflow

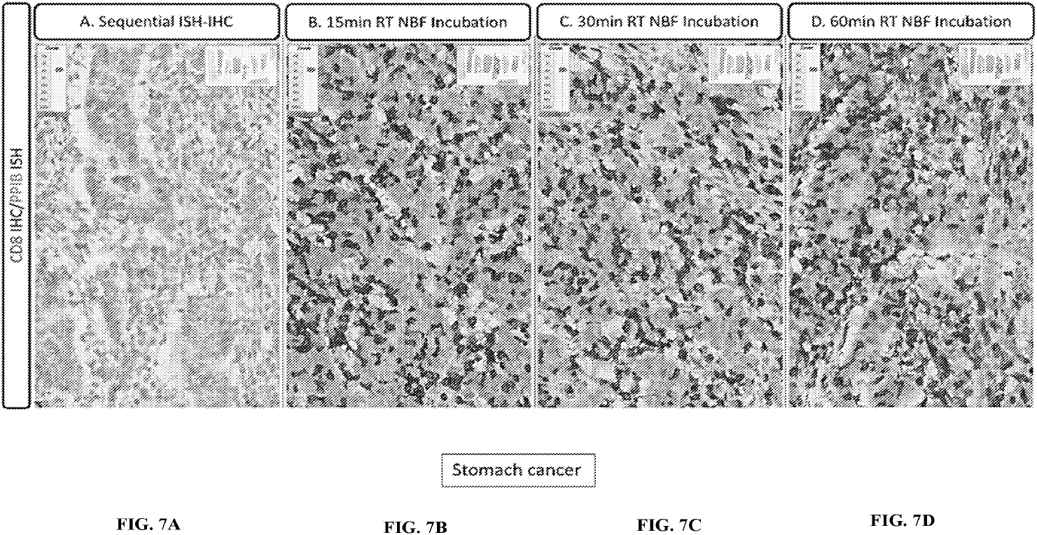

Given that crosslinking step is the key step for the ISH/IHC integrated co-detection workflow, more experiments were carried out to test the feasibility of different combinations of temperatures and durations. First, crosslinking at room temperature was shown to be effective between 15 and 60 minutes. FFPE human stomach cancer tissue was subject to ISH staining for detection of Hs-PPIB, followed immediately by IHC detection of CD8, using the RNAscope® 2.5 LS Red and Leica Bond Polymer Refine Detection kits, respectively. Using a sequential ISH-IHC co-detection workflow, Hs-PPIB ISH staining was successful, while minimal CD8 IHC signal could be detected (see FIG. 7A). Using the ISH/IHC integrated co-detection workflow, crosslinking for 15 minutes (see FIG. 7B), 30 minutes (see FIG. 7C), and 60 minutes (see FIG. 7D) at room temperature led to successful IHC detection of CD8.

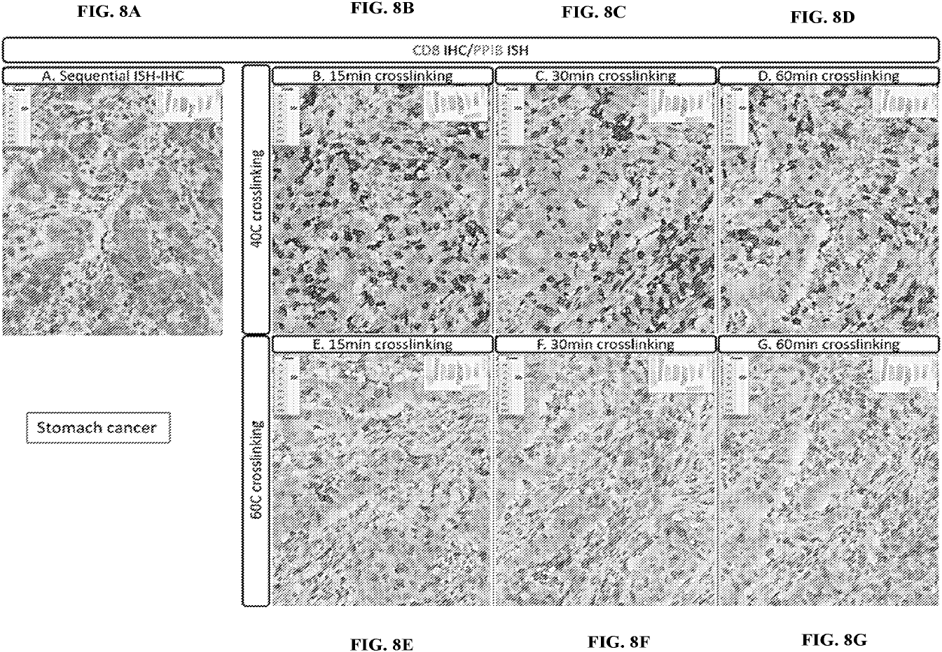

Second, crosslinking at heated temperatures was also shown to be effective between 15 and 60 minutes. FFPE human stomach cancer tissue was subject to ISH staining for detection of Hs-PPIB, followed immediately by IHC detection of CD8 as described above. Sequential ISH-IHC co-detection resulted in low CD8 IHC detection (see FIG. 8A). In contrast, crosslinking within the ISH/IHC integrated co-detection workflow at 40° C. for either 15 minutes (see FIG. 8B), 30 minutes (see FIG. 8C), or 60 minutes (see FIG. 8D) allowed for improved CD8 IHC detection. Crosslinking for either 15 minutes (see FIG. 8E), 30 minutes (see FIG. 8F), or 60 minutes (see FIG. 8G) at 60° C. also preserved IHC signal compared to sequential ISH-IHC co-detection, but to a lesser extent than the lower incubation temperature (see, e.g., FIG. 8B, FIG. 8C, and FIG. 8D).

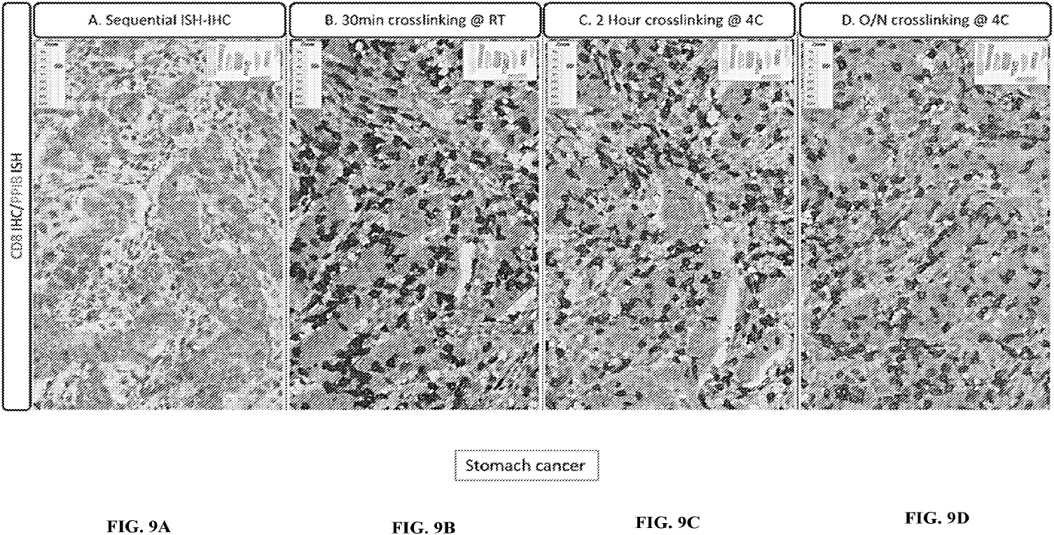

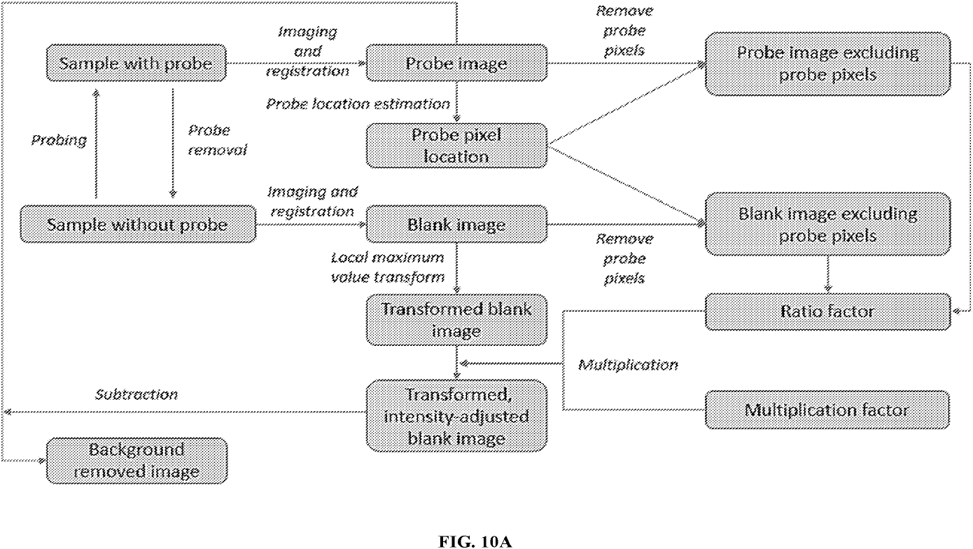

Third, crosslinking at 4° C. was also shown to be effective when performed for 2 hours or overnight. FFPE human stomach cancer tissue was subject to ISH staining for detection of Hs-PPIB, followed immediately by IHC detection of CD8 as described above. As observed previously, sequential ISH-IHC co-detection negatively impacted CD8 IHC signal (see FIG. 9A). The integrated co-detection workflow was modified to allow for crosslinking incubation below room temperature, with tissue pretreatment, antibody incubation, and crosslinking performed manually. All subsequent steps were automated on the Leica Bond Rx, including RNAscope® ISH staining and remaining IHC staining, using the RNAscope® 2.5 LS Red and Leica Bond Polymer Refine Detection kits, respectively. Compared to sequential ISH-IHC co-detection (see FIG. 9A) and crosslinking for 30 minutes at room temperature (see FIG. 9B), crosslinking for 2 hours at 4° C. (see FIG. 9C) and overnight at 4° C. (see FIG. 9D) allowed for successful IHC detection of CD8.

### 7.5 Image Processing

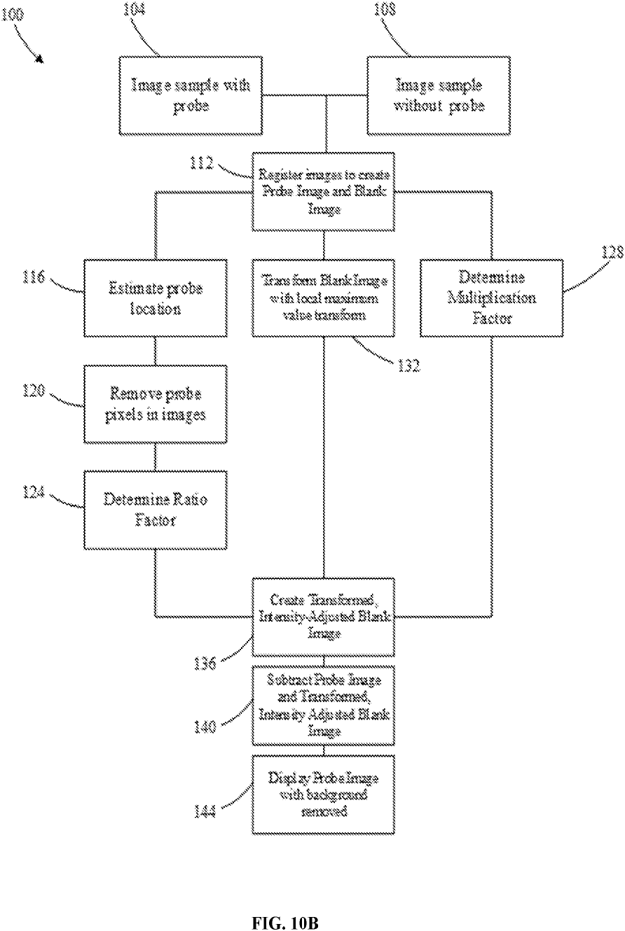

Embodiments of the present disclosure also include a method 100 for enhancing detection of a target. In some embodiments, the method 100 includes an image processing method. The method 100 is illustrated in FIG. 10B as a flowchart of steps, whereas FIG. 10A illustrates a plurality of images and corresponding factors used in the method 100 to modify the images. In the illustrated embodiment, the method 100 is implemented at least in part with a computer having corresponding instructions stored on a memory (i.e., a non-transitory computer readable medium). The final images, and in some embodiments the intermediate images, from the method 100 are stored in a memory. In some embodiments, the memory is accessible by a network. In some embodiments, user input or instructions are receivable or accessible over the network.

The method 100 includes imaging a sample with a target signal (STEP 104) to create a probe image and imaging a sample with no target signal (STEP 108) to create a background image (i.e., “blank image”). In some embodiments, the imaging utilizes a fluorescent microscope coupled to a computer via a network. In some embodiments, the target signal is obtained by subjecting the sample to a fluorescent in situ hybridization assay and/or an immunofluorescence assay. In some embodiments, the background image with no target signal is obtained by removing the target signal from the sample (i.e., by a cleaving process). In other embodiments, the background image with no target signal is obtained before the assay is performed. In other words, in some embodiments STEP 104 occurs before STEP 108 and in other embodiments STEP 104 occurs after STEP 108. In some embodiments, the target signal comprises a fluorescent label bound to a target nucleic acid. In other embodiments, the target signal comprises a fluorescent label bound to a target peptide or polypeptide.

With continued reference to FIG. 10B, the method 100 includes a STEP 112 of registering the probe image and the background image. Potential background fluorescence discrepancy between the probe image and the background image creates spatial pattern mismatches that occur due to whole sample movement between different rounds of image acquisition. To remove such discrepancies, image registration techniques (e.g., phase correlation) are utilized. Robust image registration (e.g., STEP 112) utilized detection and matching of image features to compensate for any global sample movement (i.e., translation and rotation).

The method 100 further includes modifying the background image (STEP 136) to create an adjusted background image (e.g., transformed, intensity-adjusted blank image) based on at least one image metric. As explained further herein, the at least one image metric is a ratio factor (STEPS 116, 120, 124), a multiplication factor (STEP 128), a local maximum value transform (STEP 132), and any other suitable metric. In some embodiments, the method 100 includes a single image metric. In other embodiments, the method 100 includes a combination of image metrics.

With continued reference to FIG. 10B, the method 100 further includes subtracting the adjusted background image from the probe image (STEP 140) to create a final image comprising an enhanced target signal. In other words, the modified (i.e., transformed, adjusted, scaled, etc.) blank image is used in the subtracting step (STEP 140) instead of the original blank image. In some embodiments, the enhanced target signal includes enhanced contrast. In some embodiments, the method 100 includes displaying the final image (STEP 144) on a display (e.g., a computer display). The final image may be saved to a memory and may be accessible by a user, for example, over a network. As such, the method 100 provides improved signal detection in the presence of a background with tissue autofluorescence.

In some embodiments, the image metric is a ratio factor to account for intensity differences in background between the blank image and the probe image. Intensity differences can occur when image acquisition settings are different or from photobleaching during fluorophore excitation. To compensate for background intensity differences, the method 100 includes STEPS 116, 120, and 124 to determine a ratio factor that compares the overall background intensity of the probe image versus the blank image. First, the pixel locations of the probe are estimated (STEP 116). The probe locations in the probe image are estimated using, for example, the White Top Hat algorithm (Gonzalez & Woods, 2008, Digital Image Processing), bandpass filtering (Shenoi, 2006, Introduction to Digital Signal Processing and Filter Design), or any combination of suitable methods. After determining an estimated location of the target signals in the probe image (STEP 116), the pixels at the estimated probe locations are excluded from both the probe image and the blank image (STEP 120), resulting in background-pixel-only images (i.e., background-only images). In other words, STEP 120 includes removing the estimated location from the probe image to create a first background-only image and removing the estimated location from the blank image (background image) to create a second background-only image.

Following removal of the estimated probe locations from both images (STEP 120), the method 100 includes a STEP 124 to determine a ratio factor. In other words, a statistical metric for both the probe-excluded blank image and the probe-excluded probe image is evaluated and incorporated into a ratio factor. As explained further herein, the ratio factor is utilized in some embodiments to modify the background image to create an adjusted background image (STEP 136). In other words, modifying the background image to create an adjusted background image can include, in some embodiments, scaling the background image by the ratio factor.

In some embodiments, the at least one image metric is a ratio factor of the first background-only image and the second background-only image. For example, the ratio factor in some embodiments is a first intensity to a second intensity, with the first intensity is determined from the first background-only image and the second intensity is determined from the second background-only image. In some embodiments, the first and second intensities used in the ratio factor are statistical metrics such as a statistical mean, median, or a combination of both for any portion of (including all) the intensity values in an image.

In some embodiments, the first intensity is the mean of a plurality of pixel intensity values in the first background-only image and the second intensity is the mean of a plurality of pixel intensity values in the second background-only image. In some embodiments, the mean is of all the pixel intensity values in the image. In other embodiments, the first intensity is the median of a plurality of pixel intensity values in the first background-only image, and the second intensity is the median of a plurality of pixel intensity values in the second background-only image. In some embodiments, the median is of all the pixel intensity values in the image. In another embodiment, the first intensity is the mean of a central approximately 80% of all the pixel intensity values (i.e., excluding the approximate top 10% and the approximate bottom 10%) in the first background-only image, and the second intensity is the mean of a central approximately 80% of all the pixel intensity values in the second background-only image.

In some embodiments, the image metric is a multiplication factor to account for potential local intensity differences between the blank image and the probe image. In particular, the method 100 in the illustrated embodiment includes STEP 128 to determine the multiplication factor. In some embodiments, the multiplication factor is within a range of approximately 1.0 to approximately 1.2. In other embodiments, the multiplication factor is within a range of approximately 1.0 to approximately 1.1. As explained further herein, the multiplication factor is utilized in some embodiments to modify the background image to create an adjusted background image (STEP 136). In other words, modifying the background image to create an adjusted background image can include, in some embodiments, scaling the background image by the multiplication factor.

In some embodiments, the image metric is a local maximum value transform. In particular, the method 100 in the illustrated embodiment includes STEP 132 to transform the blank image with a local maximum value transform. Even after global image registration, there may remain local background pattern mismatches that from, for example, image acquisition at different focal planes, or samples not firmly attached to the supporting material (e.g., glass slides) and partially moving between imaging sessions. To resolve this issue, local mismatches are compensated with a transform. In the illustrated embodiment, for each pixel in the blank image (“pixel of interest”), a neighborhood of a pre-defined radius surrounding the pixel of interest is searched. The search process will find the pixel of maximum intensity, and this maximum intensity is assigned to that pixel of interest. This searching procedure is performed for each pixel of interest, searching its neighborhood in the original blank image, to form a transformed blank image. As explained in greater detail herein, the transformed blank image can be used instead of the original blank image in the later subtracting step (i.e., STEP 140). In some embodiments, the pre-defined radius (“match distance”) is adjustable.

In some embodiments, the pre-defined radius used in the local maximum valve transform is within a range of approximately 0 to approximately 5 pixels. In other words, the local maximum value transform includes a search radius within a range of approximately 0 to 5 pixels. A pre-defined radius of 0 pixels is utilized, for example, when there is no noticeable local background pattern mismatch. In some embodiments, the search area is simplified to reduce computational time by using eight angularly equally spaced lines (i.e., 45 degrees apart), each with a single-pixel width, radiating from the pixel of interest.

In some embodiments, the image metric is a block-matching transform. In particular, the method 100, in some embodiments, includes a step to transform the blank image with a block-matching transform. In some embodiments, the block-matching transform is used in place of the local maximum value transform to resolve the issue of local mismatches. In some embodiments, a block (“block of interest”) is used with a pre-defined block size (e.g., a 3-pixel-by-3-pixel block). Each block in the blank image is compared with blocks of the same size in the probe image in nearby locations (i.e., within a pre-defined block search size). The search determines the nearby block that is most similar to the block of interest. A similarity metric is utilized to measure the similarity of the blocks, and the searched nearby block with the highest similarity metric is determined to be the target block. Then, the block of interest is moved to the corresponding location of the target block. In some embodiments, the similarity metric is a mean absolute difference, a sum of absolute difference, a mean squared difference, or a sum of squared difference, wherein the differences are the pixel intensity differences between the two blocks being compared. As such, the block-matching transform is performed for each block of interest, searching its corresponding neighborhood in the probe image and moving its location accordingly, to form a transformed blank image. In some embodiments, this transformed blank image is used instead of the original blank image in later subtracting steps (i.e., STEP 140).

In some embodiments, the pre-defined block size and the pre-defined block search size are adjustable. In some embodiments, the pre-defined block size used in the block-matching transform is within a range of approximately 1 to approximately 10 pixels. In other words, the block-matching transform includes a block size within a range of approximately 1 to 10 pixels. In some embodiments, the pre-defined block search size used in the block matching transform is within a range of approximately 1 to approximately 10 pixels. In other words, the block-matching transform includes a block search size within a range of approximately 1 to 10 pixels.

In some embodiments, the method for enhancing detection of a target includes any combination of the steps described herein, in various orders. In some embodiments, steps may be omitted. Further, the order of the steps may be reversed, altered, or performed simultaneously.

In at least one embodiment, the electronic-based aspects of the method 100 may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A method for preparing a biological sample for simultaneously detecting a target nucleic acid and a target protein, the method comprising:
(i) incubating the biological sample with a primary antibody;
(ii) treating the biological sample with a crosslinking agent after (i); and
(iii) detecting the target nucleic acid by in situ hybridization after (ii).

2. The method of claim 1, further comprising treating the biological sample with a protease after treating the biological sample with the crosslinking agent (ii) and before detecting the target nucleic acid by in situ hybridization (iii).

3. The method of claim 1 or claim 2, further comprising incubating the biological sample with a secondary antibody or other labeling methods after detecting the target nucleic acid by in situ hybridization (iii).

4. The method of any one of claims 1 to 3, wherein the target nucleic acid is RNA.

5. The method of any one of claims 1 to 3, wherein the target nucleic acid is DNA.

6. The method of any one of claims 1 to 5, wherein the biological sample is a tissue specimen or is derived from a tissue specimen.

7. The method of any one of claims 1 to 5, wherein the biological sample is a blood sample or is derived from a blood sample.

8. The method of any one of claims 1 to 5, wherein the biological sample is a cytological sample or is derived from a cytological sample.

9. The method of any one of claims 1 to 5, wherein the biological sample is cultured cells or a sample containing exosomes.

10. The method of any one of claims 1 to 9, wherein the crosslinking agent in step (ii) is a fixative.

11. The method of claim 10, wherein the fixative is neutral buffered formalin.

12. The method of claim 11, wherein the neutral buffered formalin is 10% neutral buffered formalin.

13. The method of any one of claims 1 to 12, wherein the step of treating the biological sample with the crosslinking agent lasts for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

14. The method of any one of claims 1 to 12, wherein the step of treating the biological sample with the crosslinking agent is performed at about 4° C., room temperature, about 40° C., or about 60° C.

15. The method of any one of claims 1 to 12, wherein the step of treating the biological sample with the crosslinking agent is performed at about 4° C. for about 2 hours.

16. The method of any one of claims 1 to 12, wherein the step of treating the biological sample with the crosslinking agent is performed at about 4° C. for about 16 to about 18 hours.

17. The method of any one of claims 1 to 12, wherein the step of treating the biological sample with the crosslinking agent is performed at room temperature for about 15 minutes.

18. The method of any one of claims 1 to 12, wherein the step of treating the biological sample with the crosslinking agent is performed at room temperature for about 30 minutes.

19. The method of any one of claims 1 to 12, wherein the step of treating the biological sample with the crosslinking agent is performed at room temperature for about 60 minutes.

20. The method of any one of claims 1 to 12, wherein the step of treating the biological sample with the crosslinking agent is performed at about 40° C. for about 15 minutes.

21. The method of any one of claims 1 to 12, wherein the step of treating the biological sample with the crosslinking agent is performed at about 40° C. for about 30 minutes.

22. The method of any one of claims 1 to 12, wherein the step of treating the biological sample with the crosslinking agent is performed at about 40° C. for about 60 minutes.

23. The method of any one of claims 1 to 12, wherein the step of treating the biological sample with the crosslinking agent is performed at about 60° C. for about 15 minutes.

24. The method of any one of claims 1 to 12, wherein the step of treating the biological sample with the crosslinking agent is performed at about 60° C. for about 30 minutes.

25. The method of any one of claims 1 to 12, wherein the step of treating the biological sample with the crosslinking agent is performed at about 60° C. for about 60 minutes.

26. A method for simultaneously detecting a target nucleic acid and a target protein in a biological sample, the method comprising:
(i) incubating the biological sample with a primary antibody;
(ii) treating the biological sample with a crosslinking agent;
(iii) treating the biological sample with a protease;
(iv) detecting the target nucleic acid by in situ hybridization; and
(v) detecting the target protein by incubating the biological sample with a secondary antibody or other labeling methods.

27. The method of claim 26, wherein the target nucleic acid is RNA.

28. The method of claim 26, wherein the target nucleic acid is DNA.

29. The method of any one of claims 26 to 28, wherein the step of detecting the target nucleic acid by in situ hybridization comprises:
(i) providing one or more target probe(s) capable of hybridizing to the target nucleic acid;
(ii) providing a signal-generating complex capable of hybridizing to the one or more target probe(s), wherein the signal-generating complex comprises a nucleic acid component capable of hybridizing to the one or more target probe(s) and a label probe;
(iii) hybridizing the target nucleic acid to the one or more target probe(s); and
(iv) capturing the signal-generating complex to the one or more target probe(s) and thereby capturing the signal-generating complex to the target nucleic acid.

30. The method of claim 29, wherein each of the one or more target probe(s) comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

31. The method of any one of claims 26 to 30, wherein the method further comprises providing an immunohistochemistry label capable of binding to the secondary antibody for detecting the target protein; or wherein the secondary antibody is pre-labeled.

32. The method of any one of claims 26 to 31, wherein the biological sample is a tissue specimen or is derived from a tissue specimen.

33. The method of any one of claims 26 to 31, wherein the biological sample is a blood sample or is derived from a blood sample.

34. The method of any one of claims 26 to 31, wherein the biological sample is a cytological sample or is derived from a cytological sample.

35. The method of any one of claims 26 to 31, wherein the biological sample is cultured cells or a sample containing exosomes.

36. The method of claim 26, wherein the crosslinking agent is a fixative.

37. The method of claim 36, wherein the fixative is neutral buffered formalin.

38. The method of claim 37, wherein the neutral buffered formalin is 10% neutral buffered formalin.

39. The method of any one of claims 26 to 38, wherein the step of treating the biological sample with the crosslinking agent lasts for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

40. The method of any one of claims 26 to 38, wherein the step of treating the biological sample with the crosslinking agent is performed at about 4° C., room temperature, about 40° C., or about 60° C.

41. The method of any one of claims 26 to 38, wherein the step of treating the biological sample with the crosslinking agent is performed at about 4° C. for about 2 hours.

42. The method of any one of claims 26 to 38, wherein the step of treating the biological sample with the crosslinking agent is performed at about 4° C. for about 16 to about 18 hours.

43. The method of any one of claims 26 to 38, wherein the step of treating the biological sample with the crosslinking agent is performed at room temperature for about 15 minutes.

44. The method of any one of claims 26 to 38, wherein the step of treating the biological sample with the crosslinking agent is performed at room temperature for about 30 minutes.

45. The method of any one of claims 26 to 38, wherein the step of treating the biological sample with the crosslinking agent is performed at room temperature for about 60 minutes.

46. The method of any one of claims 26 to 38, wherein the step of treating the biological sample with the crosslinking agent is performed at about 40° C. for about 15 minutes.

47. The method of any one of claims 26 to 38, wherein the step of treating the biological sample with the crosslinking agent is performed at about 40° C. for about 30 minutes.

48. The method of any one of claims 26 to 38, wherein the step of treating the biological sample with the crosslinking agent is performed at about 40° C. for about 60 minutes.

49. The method of any one of claims 26 to 38, wherein the step of treating the biological sample with the crosslinking agent is performed at about 60° C. for about 15 minutes.

50. The method of any one of claims 26 to 38, wherein the step of treating the biological sample with the crosslinking agent is performed at about 60° C. for about 30 minutes.

51. The method of any one of claims 26 to 38, wherein the step of treating the biological sample with the crosslinking agent is performed at about 60° C. for about 60 minutes.

52. The method of any one of claims 1 to 51, wherein the method is used for mapping spatial organization in a complex tissue, and optionally wherein the complex tissue is a tumor tissue.

53. The method of any one of claims 1 to 51, wherein the method is used for detecting altered gene expression in the biological samples from a diseased model.

54. The method of any one of claims 1 to 51, wherein the method is used for validating novel antibodies.

55. A kit for simultaneously detecting a target nucleic acid and a target protein in a biological sample, comprising:
(i) a crosslinking agent; and
(ii) an instruction indicating that the crosslinking agent is used after incubating the biological sample with a primary antibody that detects the target protein.

56. The kit of claim 55, further comprising a protease.

57. A kit for simultaneously detecting a target nucleic acid and a target protein in a biological sample, comprising:
(i) a crosslinking agent; and
(ii) a protease.

58. The kit of claim 57, further comprising an instruction indicating that the crosslinking agent is used before the protease and the crosslinking agent is used after a primary antibody that detects the target protein.

59. The kit of any one of claims 55 to 58, further comprising an agent for detecting the target nucleic acid and/or an agent for detecting the target protein.

60. The kit of any one of claims 55 to 59, wherein the target nucleic acid is RNA.

61. The kit of any one of claims 55 to 59, wherein the target nucleic acid is DNA.

62. The kit of any one of claims 55 to 61, wherein the instruction further indicates treating the biological sample with the crosslinking agent for about 15 minutes, about 30 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

63. The kit of any one of claims 55 to 61, wherein the instruction further indicates treating the biological sample with the crosslinking agent at about 4° C., room temperature, about 40° C., or about 60° C.

64. The kit of any one of claims 55 to 61, wherein the instruction further indicates treating the biological sample with the crosslinking agent at about 4° C. for about 2 hours.

65. The kit of any one of claims 55 to 61, wherein the instruction further indicates treating the biological sample with the crosslinking agent at about 4° C. for about 16 to about 18 hours.

66. The kit of any one of claims 55 to 61, wherein the instruction further indicates treating the biological sample with the crosslinking agent at room temperature for about 15 minutes.

67. The kit of any one of claims 55 to 61, wherein the instruction further indicates treating the biological sample with the crosslinking agent at room temperature for about 30 minutes.

68. The kit of any one of claims 55 to 61, wherein the instruction further indicates treating the biological sample with the crosslinking agent at room temperature for about 60 minutes.

69. The kit of any one of claims 55 to 61, wherein the instruction further indicates treating the biological sample with the crosslinking agent at about 40° C. for about 15 minutes.

70. The kit of any one of claims 55 to 61, wherein the instruction further indicates treating the biological sample with the crosslinking agent at about 40° C. for about 30 minutes.

71. The kit of any one of claims 55 to 6 1, wherein the instruction further indicates treating the biological sample with the crosslinking agent at about 40° C. for about 60 minutes.

72. The kit of any one of claims 55 to 61, wherein the instruction further indicates treating the biological sample with the crosslinking agent at about 60° C. for about 15 minutes.

73. The kit of any one of claims 55 to 61, wherein the instruction further indicates treating the biological sample with the crosslinking agent at about 60° C. for about 30 minutes.

74. The kit of any one of claims 55 to 6 1, wherein the instruction further indicates treating the biological sample with the crosslinking agent at about 60° C. for about 60 minutes.

75. The kit of claim 59, wherein the agent for detecting the target nucleic acid comprises one or more target probe(s) capable of hybridizing to the target nucleic acid; and a signal-generating complex capable of hybridizing to the one or more target probe(s), wherein said signal-generating complex comprises a nucleic acid component capable of hybridizing to the one or more target probe(s) and a label probe.

76. The kit of claim 75, wherein each of the one or more target probe(s) comprises a target (T) section and a label (L) section, wherein the T section is a nucleic acid sequence complementary to a section on the target nucleic acid and the L section is a nucleic acid sequence complementary to a section on the nucleic acid component of the signal generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

77. The kit of any one of claims 55 to 76, further comprising a tool for obtaining the biological sample.

78. The kit of claim 77, wherein the biological sample is a tissue specimen or is derived from a tissue specimen.

79. The kit of claim 77, wherein the biological sample is a blood sample or is derived from a blood sample.

80. The kit of claim 77, wherein the biological sample is a cytological sample or is derived from a cytological sample.

81. The kit of claim 77, wherein the biological sample is cultured cells or a sample containing exosomes.

82. The kit of any one of claims 55 to 81, wherein the kit is used for mapping spatial organization in a complex tissue, and optionally wherein the complex tissue is a tumor tissue.

83. The kit of any one of claims 55 to 81, wherein the kit is used for detecting altered gene expression in the biological samples from a diseased model.

84. The kit of any one of claims 55 to 81, wherein the kit is used for validating novel antibodies.

85. A biological sample prepared according to the method of any one of claims 1 to 25.
